(19) United States
(12) Patent Application Publication
(10) Pub. No.: US 2025/0305067 A1
(43) Pub. Date: Oct. 2, 2025

(54) DNA-METHYLATION DETECTION IN ANIMAL-DERIVED PRODUCTS

(71) Applicant: Evonik Operations GmbH, Essen (DE)

(72) Inventors: Rose WHELAN, Ambrosden Oxfordshire (GB); Sanjanaa NAGARAJAN, Singapore (SG); Suki ROY, Sydney, New South Wales (AU); Florian BÖHL, Neckargemünd (DE); Lingzhi HUANG, Singapore (SG); Kit Yeng WONG, Singapore (SG); Sarah CHAN, Bellevue, WA (US); Daniel FRANKE, Essen (DE)

(73) Assignee: Evonik Operations GmbH, Essen (DE)

(21) Appl. No.: 19/107,661

(22) PCT Filed: Aug. 23, 2023
(86) PCT No.: PCT/EP2023/073124

§ 371 Date: Feb. 28, 2025

(30) Foreign Application Priority Data
Sep. 1, 2022 (EP) 22193450.8

(51) Int. Cl. C12Q 1/6888; C12Q 1/6809

(52) U.S. Cl. CPC C12Q 1/6888; C12Q 1/6809; C12Q 2600/124; C12Q 2600/136

(57) ABSTRACT

The present invention is related to a method of detecting DNA methylation and/or determining a test methylation profile from genomic material contained in a biological sample obtained from a test animal-derived product, the method comprises the step of: contacting a genomic material sample from the test animal-derived product with a DNA methylation array specific for species of the test animal, wherein the test animal is a monogastric livestock.

### CROSS-REFERENCE TO RELATED APPLICATIONS

This is a National Stage of International Application No. PCT/EP2023/073124 filed Aug. 23, 2023, claiming priority based on European Patent Application No. 22193450.8 filed Sep. 1, 2022.

### FIELD OF THE INVENTION

The present invention relates to a method of detecting DNA-methylation in farm animal derived products. In particular, the method is a DNA array-based method for detecting DNA methylation, in products that are derived from monogastric livestock. The DNA array-based method may be used to determine a methylation profile for the animal derived product which can then be used in several applications which determine the origin and welfare of the animal and also if the animal has been exposed to antibiotics and the like and/or the means of having slaughtered the animal.

### BACKGROUND OF THE INVENTION

As income and education levels rise, consumers have been shown to take a greater interest in the quality of food they are eating and are increasingly more willing to pay premiums for meat products with assurance of quality, safety, sustainability, and high animal welfare conditions (Wu et al. Foods 2021, 10, 2490). Many of these qualities cannot be visually assessed and therefore control of food via regulatory bodies like the European Food and Drug Safety Administration and the Food and Drug Administration (FDA) of the United States of America improve consumer trust. Clear, simple food-labelling from independent certification bodies adds additional level of assurance to consumers. Of the food labelling qualities possible on meat products, geographical origin was shown to be valued by all consumers regardless of culture (Wu 2021). Additionally, a study of 10,000 consumers over multiple countries (Japan, USA, Germany, China and Thailand) indicated that labels are trusted more when certified by scientific experts compared to claims made by producers, retailers or even governing bodies (Rupprecht et al. Food and Chemical Toxicology 137 (2020) 111170).

Traceability of origin via bar codes, QR codes or online links printed on packaging has also been shown to further improve consumer trust and potentially the premiums consumers are willing to pay for assurance of the origin of their meat products. Currently, traceability technologies involve data recording and reporting that may be supported by computing, artificial intelligence and/or decentralized blockchain. As the current systems mostly rely on human reporting chains from farm to fork, there are many possibilities for misinformation to be introduced due to error or intentional fraud.

A single food fraud event may incur significant loss of consumer trust in a brand, recalls, lawsuits, lost revenue and in severe cases even criminal charges (Esteki et al. Comprehensive Reviews in Food Science and Food Safety Vol. 18, 2019). Many countries such as the USA's FDA have specific criterion determining when food fraud can be considered a criminal offense (Jurica et al. Foods 2021, 10, 2570). However, despite the potential loss of profits and legal frameworks criminalizing food fraud, intentional economically motivated adulteration or mislabeling of food products continues to be an issue worldwide. To better support consumer trust in high value meat products, there is a need for scientific, evidence-based tests that reliably and consistently offer assurance of how and where farm animals such as livestock and poultry were raised.

To date, there are several methods available for analyses of meat products. For example, origin traceability technologies have been created by measuring isotope-ratios via mass spectrometry (Zhao et al. Food Chemistry 145 (2014) 300-305) or determining the trace element fingerprint in a sample. Additionally, the confirmation of species from meat products can be accomplished with PCR-based technologies. However, multiple technologies need to be utilized on a single sample to get adequate information about origin and species of meat samples, which adds unnecessary cost and time to verification process for these food products. To our knowledge there are currently no reliable technologies for meat testing that are able to determine the health status of livestock nor the welfare standards by which an animal was reared prior to slaughter. Therefore, reliable, evidence-based technologies for determining multiple factors of meat quality are sought for the assurance of origin, quality, welfare and sustainability of animal-derived food products.

Epigenetics technologies may provide a solution for the unequivocal, multi-target analysis of meat. DNA methylation is one of the best understood mechanisms of epigenetics and is known to be altered by various aspects the environmental conditions an animal is exposed to. Therefore, methylation patterns on the genome can be used to differentiate healthy from inflamed animals, for example (Raddatz et al. Communications Biology, 4:76 (2021)). WO2022/023208 also discloses DNA methylation being used at least to determine the geographical origin of an animal. Traditionally, global methylation patterns especially for non-human species have been assessed from extracted DNA from different tissue and/or cells, by using whole genome bisulfite sequencing (WGBS) or reduced representation bisulfite sequencing (RRBS). Both approaches first use a bisulfite treatment step to convert all unmethylated cytosine nucleotides in the genome to uracil, leaving methylated and hemi-methylated cytosine nucleotides unchanged (Stevens et al. Genome Res. 2013. 23: 1541-1553). Next, generation sequencing is performed, and sequences generated are processed (aligned to reference genomes) and analysed to indicate methylation differences at individual CpG sites. WGBS covers the CpG sites on the whole genome, while RRBS covers only 3-4% of all methylation sites of a genome but represents 85% of CpG sites of the dynamically methylated regions (Illumina Field Guide to Methylation Methods, 2016). These technologies while highly informative are costly, time-consuming, and computationally intensive, prohibiting fast turnaround times.

Accordingly, there is a need in the art for an improved a high-throughput, cost-effective, reliable, efficient and robust method to determine the origin and welfare standards by which an animal was reared through a small sample of DNA.

### DESCRIPTION OF THE INVENTION

The present invention solves the problems above by providing an array-based method to determine origin, welfare and several other rearing conditions of organisms by using DNA-methylation profiling. DNA-Methylation-based arrays allow for a high-throughput and robust method to determine semi-quantitative/quantitative DNA-methylation information through a small sample of extracted DNA of interest. These custom designed arrays may use Illumina iScan and Infinium platform technology or an equivalent thereof, which allows on each chip for example 100,000 different bead types that covalently bind DNA-methylation probes. Each probe represents one CpG Methylation site at the end of the probe sequence. DNA samples undergo bisulfite conversion, amplification, fragmentation, precipitation and resuspension steps before hybridization on an array chip. Once on the chip the DNA hybridizes to the beads for each CpG site so that methylation changes at each site can be detected specifically through single nucleotide extension. This is especially advantageous as the array-based method is simple and the results of the methylation-based array are accurate and reproducible.

Further, compared to traditional sequencing which can take weeks to generate data, the array technology has a much shorter turn-around time. The volume and complexity of data generated is lesser compared to sequencing making it computationally less intensive. This allows for quicker computation to achieve interpretable results from experimental groups. Overall microarray technology is roughly 10× faster and 10× cheaper than traditional sequencing while still quantifiable for the methylation level at specific CpG sites. Methylation-array technology therefore offers a fast and flexible system that can be used for many applications, allowing for the scalability of epigenetics research, and commercialization of DNA-methylation based solutions for along the food value chains.

As methylation changes on CpG sites can occur due to different environmental conditions, methylation patterns in the genome can be used to determine aspects of how and where an animal was raised, for example geographical location, health, and certain rearing standards that may determine meat categorizations like high animal welfare, organic, kosher or halal. The DNA array-based method according to any aspect of the present invention includes probes that bind specific CpG sites with known methylation changes from different locations and standards of rearing, and also from promotors, from candidate genes with emphasis on immune system genes, feed-linked, genes, antibiotics linked genes, breast muscle development genes (Myopathy) etc. The method according to any aspect of the present invention may be used to determine origin, welfare, and other meat quality aspects scientifically and unequivocally.

The term ‘epigenetic change’ as used herein refers to a chemical (e.g., methylation) change or protein (e.g., histones) change that takes place to a gene body or a promoter thereof. Through epigenetic changes, environmental factors like. diet, stress and prenatal nutrition can make an imprint on genes passed from one generation to the next.

According to one aspect of the present invention, there is provided a method of detecting and/or quantifying DNA methylation from genomic material contained in a biological sample obtained from a test animal-derived product, the method comprises the step of:
contacting a genomic material sample from the test animal-derived product with a DNA methylation array specific for species of the test animal,
   wherein the test animal is a monogastric livestock.

The term “array” as used herein refers to an intentionally created collection of probe molecules which can be prepared either synthetically or biosynthetically. The probe molecules in the array can be identical or different from each other. The array can assume a variety of formats, for example, libraries of soluble molecules; libraries of compounds tethered to resin beads, silica chips, or other solid supports.

In particular, a DNA methylation-based array also known as a DNA methylation array or a chip provides a convenient platform for simultaneous analysis of large numbers of CpG sites, for example, at least 2, 3, 4, 5, 6, 7, 8, 9, 10, 50, 100, 500, 1000, 5000, 10,000, 100,000 or more sites or loci. In particular, the array comprises a plurality of different probe molecules that can be attached to a substrate or otherwise spatially distinguished in an array. Examples of arrays that may be used according to any aspect of the present invention include slide arrays, silicon wafer arrays, liquid arrays, bead-based arrays and the like. In one example, array technology used according to any aspect of the present invention combines a miniaturized array platform, a high level of assay multiplexing, and scalable automation for sample handling and data processing.

In particular, the array according to any aspect of the present invention may be an array of arrays, also referred to as a composite array, having a plurality of individual arrays that is configured to allow processing of multiple samples simultaneously. Examples of composite arrays and the technology behind them are disclosed at least in U.S. Pat. No. 6,429,027 and US 2002/0102578. A substrate of a composite array may include a plurality of individual array locations, each having a plurality of probes, and each physically separated from other assay locations on the same substrate such that a fluid contacting one array location is prevented from contacting another array location. Each array location can have a plurality of different probe molecules that are directly attached to the substrate or that are attached to the substrate via rigid particles in wells (also referred to herein as beads in wells).

In one example, an array substrate can be a fibre optical bundle or array of bundles as described in U.S. Pat. Nos. 6,023,540, 6,200,737 and/or 6,327,410. An optical fibre bundle or array of bundles can have probes attached directly to the fibres or via beads. A skilled person would be able to easily determine which substrate will be most suitable for the array according to any aspect of the present invention. WO2004110246 further discloses other substrates and methods of attaching beads to the substrates that may be used in the array according to any aspect of the present invention.

In one example, a surface of the substrate may have physical alterations to enable the attachment of probes or produce array locations. For example, the surface of a substrate can be modified to contain chemically modified sites that are useful for attaching, either-covalently or non-covalently, probe molecules or particles having attached probe molecules. Probes may be attached using any of a variety of methods known in the art including, an ink-jet printing method, a spotting technique, a photolithographic synthesis method, or printing method utilizing a mask. WO2004110246 discloses these techniques in more detail.

In one example, the DNA methylation-based array according to any aspect of the present invention may be a bead-based array, where the beads are associated with a solid support such as those commercially available from Illumina, Inc. (San Diego, Calif.). An array of beads useful according to any aspect of the present invention can also be in a fluid format such as a fluid stream of a flow cytometer or similar device. Commercially available fluid formats for distinguishing beads include, for example, those used in XMAP™ technologies from Luminex or MPSS™ methods from Lynx Therapeutics.

In another example, the DNA methylation-based array according to any aspect of the present invention may further comprise
at least one probe molecule specific for at least one single nucleotide polymorphism (SNP) of the first species of animal; and
at least one probe molecule specific for at least one SNP of the second species of animal.

These probes specific for SNPs may be used for SNP genotyping, which is the measurement of genetic variations of SNPs between members of a species. In particular, an SNP is a single base pair mutation at a specific locus, usually consisting of two alleles (where the rare allele frequency is >1%) that are conserved during evolution. These probes enable the identification of a species, particularly breed of a species. In particular, when a DNA sample is introduced to the array according to any aspect of the present invention, these probes specific to SNPs can be used to determine if the sample is from the first and/or second species of animal found on the array and whether there is DNA from another species other than the first and second animal species that has contaminated the DNA sample.

The term “solid support”, “support”, and “substrate” as used herein are used interchangeably and refer to a material or group of materials having a rigid or semi-rigid surface or surfaces. In many examples, at least one surface of the solid support will be substantially flat, although in some examples it may be desirable to physically separate synthesis regions for different compounds with, for example, wells, raised regions, pins, etched trenches, or the like.

The DNA methylation array according to any aspect of the present invention may be a very high-density array, for example, those having from about 10,000,000 probes/cm² to about 2,000,000,000 probes/cm² or from about 100,000,000 probes/cm² to about 1,000,000,000 probes/cm². High density arrays are especially useful according to any aspect of the present invention for including the multitude of CpG sites from the different species on the array.

The DNA methylation array according to any aspect of the present invention may be used to analyse or evaluate such pluralities of loci simultaneously or sequentially as desired. In one example, a plurality of different probe molecules can be attached to a substrate or otherwise spatially distinguished in an array. Each probe is typically specific for a particular locus and can be used to distinguish methylation state of the locus.

The term “probe molecules” as used herein refers to a surface-immobilized molecule that can be recognized by a particular target. Probes used in the array can be specific for the methylated allele of a CpG site, the non-methylated allele of the CpG site or both.

The term “target” as used herein refers to a molecule that has an affinity for a given probe molecule. Targets may be naturally occurring or man-made molecules. Also, they can be employed in their unaltered state or as aggregates with other species. Targets may be attached, covalently or noncovalently, to a binding member, either directly or via a specific binding substance. Examples of targets which can be employed according to any aspect of the present invention are methylated and non-methylated CpG sites. Targets are sometimes referred to in the art as anti-probes. As the term targets is used herein, no difference in meaning is intended.

In particular, the probe molecule according to any aspect of the present invention comprises a nucleic acid sequence that is complementary to a distinct CpG site. The array according to any aspect of the present invention thus comprises several distinct or unique locations, wherein each location comprises a specific probe molecule that is complementary to a distinct CpG site of an animal species. The array thus comprises a plurality of locations, each location with a specific probe molecule that is complementary to a distinct CpG site of an animal species. In particular, the array according to any aspect of the present invention, comprises distinct locations, where each location comprises a specific probe molecule that is complementary to a distinct CpG site of at least two animal species. The array according to any aspect of the present invention thus comprises distinct locations with specific probe molecules where each probe molecule is complementary to a distinct CpG site from at least two animal species.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Perfectly complementary refers to 100% complementarity over the length of a sequence. For example, a 25 base probe is perfectly complementary to a target when all 25 bases of the probe are complementary to a contiguous 25 base sequence of the target with no mismatches between the probe and the target over the length of the probe.

As used herein, a “CpG site” or “methylation site” is a nucleotide within a nucleic acid (DNA or RNA) that is susceptible to methylation either by natural occurring events in vivo or by an event instituted to chemically methylate the nucleotide in vitro. Some of these sites may be hypermethylated and some may be hypomethylated in a cell.

As used herein, a “methylated nucleic acid molecule” refers to a nucleic acid molecule that contains one or more nucleotides that is/are methylated.

As used herein, a “methylated nucleotide” or a “methylated nucleotide base” refers to the presence of a methyl moiety on a nucleotide base, where the methyl moiety is usually not present in a recognized typical nucleotide base. For example, cytosine in its usual form does not contain a methyl moiety on its pyrimidine ring, but 5-methylcytosine contains a methyl moiety at position 5 of its pyrimidine ring. Therefore, cytosine in its usual form may not be considered a methylated nucleotide and 5-methylcytosine may be considered a methylated nucleotide. In another example, thymine may contain a methyl moiety at position 5 of its pyrimidine ring, however, for purposes herein, thymine may not be considered a methylated nucleotide when present in DNA. Typical nucleotide bases for DNA are thymine, adenine, cytosine and guanine. Typical bases for RNA are uracil, adenine, cytosine and guanine. Correspondingly a “methylation site” is the location in the target gene nucleic acid region where methylation has the possibility of occurring. For example, a location containing CpG is a methylation site wherein the cytosine may or may not be methylated. In particular, the term “methylated nucleotide” refers to nucleotides that carry a methyl group attached to a position of a nucleotide that is accessible for methylation. These methylated nucleotides are usually found in nature and to date, methylated cytosine that occurs mostly in the context of the dinucleotide CpG, but also in the context of CpNpG- and CpNpN-sequences may be considered the most common. In principle, other naturally occurring nucleotides may also be methylated but they will not be taken into consideration with regard to any aspect of the present invention.

A “CpG island” as used herein describes a segment of DNA sequence that comprises a functionally or structurally deviated CpG density. For example, Yamada et al. have described a set of standards for determining a CpG island: it must be at least 400 nucleotides in length, has a greater than 50% GC content, and an OCF/ECF ratio greater than 0.6 (Yamada et al., 2004, Genome Research, 14, 247-266). Others have defined a CpG island less stringently as a sequence at least 200 nucleotides in length, having a greater than 50% GC content, and an OCF/ECF ratio greater than 0.6 (Takai et al., 2002, Proc. Natl. Acad. Sci. USA, 99, 3740-3745). In context of the present invention, the terms “methylation profile”, “methylation pattern”, “methylation state” or “methylation status,” are used herein to describe the state, situation or condition of methylation of a genomic sequence, and such terms refer to the characteristics of a DNA segment at a particular genomic locus in relation to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles.

The term “methylation status” refers to the status of a specific methylation site (i.e. methylated vs. non-methylated) which means a residue or methylation site is methylated or not methylated. Then, based on the methylation status of one or more methylation sites, a methylation profile may be determined.

The term “methylation level” refers to the level of a specific methylation site which can range from 0 (=unmethylated) to 1 (=fully methylated). Thus, based on the methylation level of one or more methylation sites, a methylation profile may be determined. Accordingly, the term “methylation” profile” or also “methylation pattern” refers to the relative or absolute concentration of methylated C or unmethylated C at any particular stretch of residues in a biological sample. For example, if cytosine (C) residue(s) not typically methylated within a DNA sequence are more methylated in a sample, it may be referred to as “hypermethylated”; whereas if cytosine (C) residue(s) typically methylated within a DNA sequence are less methylated, it may be referred to as “hypomethylated”. Likewise, if the cytosine (C) residue(s) within a DNA sequence (e.g., sample nucleic acid) are more methylated when compared to another sequence from a different region or from a different individual (e.g., relative to normal nucleic acid), that sequence is considered hypermethylated compared to the other sequence. Alternatively, if the cytosine (C) residue(s) within a DNA sequence are less methylated as compared to another sequence from a different region or from a different individual, that sequence is considered hypomethylated compared to the other sequence. These sequences are said to be “differentially methylated”. For example, when the methylation status differs between inflamed and non-inflamed tissues, the sequences are considered “differentially methylated”. Measurement of the levels of differential methylation may be done by a variety of ways known to those skilled in the art. One method is to measure the methylation level of individual interrogated CpG sites determined by the bisulfite sequencing method, as a non-limiting example.

Bisulfite treatment’ of genomic DNA used interchangeably with the term ‘bisulfite modification’, refers to the treatment of the genomic DNA with a deaminating agent such as a bisulfite that may be used to treat all DNA, methylated or not. In particular, the term “bisulfite” as used herein encompasses any suitable type of bisulfite, such as sodium bisulfite, or other chemical agents that are capable of chemically converting a cytosine (C) to an uracil (U) without chemically modifying a methylated cytosine and therefore can be used to differentially modify a DNA sequence based on the methylation status of the DNA, e.g., U.S. Pat. Pub. US 2010/0112595. As used herein, a reagent that “differentially modifies” methylated or non-methylated DNA encompasses any reagent that modifies methylated and/or unmethylated DNA in a process through which distinguishable products result from methylated and non-methylated DNA, thereby allowing the identification of the DNA methylation status. Such processes may include, but are not limited to, chemical reactions (such as a C to U conversion by bisulfite) and enzymatic treatment (such as cleavage by a methylation-dependent endonuclease). Thus, an enzyme that preferentially cleaves or digests methylated DNA is one capable of cleaving or digesting a DNA molecule at a much higher efficiency when the DNA is methylated, whereas an enzyme that preferentially cleaves or digests unmethylated DNA exhibits a significantly higher efficiency when the DNA is not methylated.

Accordingly, before step (a) according to any aspect of the present invention is carried out, the genomic DNA contained/obtained or extracted from the cell, is first bisulfite treated.

An alternative method available in the art may be used instead of bisulfite treatment. A skilled person will understand which other methods to use. In one example, TET-assisted pyridine borane sequencing (TAPS) may be used for detection of 5mC and 5hmC (Yibin Liu, et al., Nature Biotechnology, 37: 424-429 (2019).

As used herein, the term “genomic material” refers to nucleic acid molecules or fragments of the genome of the animal according to any aspect of the present invention. In particular, such nucleic acid molecules or fragments are DNA or RNA or hybrids thereof, and most preferably are molecules of the DNA genome of a subject or group of subjects.

The term ‘biological sample’ as used herein may be selected from the group consisting of muscle, organ tissue, milk, blood, brain, sperm and any other tissue or sample that provides genomic DNA to be used in the method according to any aspect of the present invention. In particular, the biological sample may comprise any biological material obtained from the subject that contains DNA, and may be liquid, solid or both, may be tissue or bone, or a body fluid such as blood, lymph, etc. In particular, the biological sample useful for the present invention may comprise biological cells or fragments thereof.

As used herein, the “DNA sample” refers to the DNA extracted from a cell of the animal according to any aspect of the present invention using known methods in the art.

As used herein, the term ‘animal-derived product’ refers to products that originate from animals. In particular, the term ‘test animal-derived product’ refers to the sample or subject in question that is to be introduced to the array according to any aspect of the present invention. These products from animals may include meat and meat products, also including fat, flesh, blood, processed meat, and lesser-known products, such as isinglass and rennet, poultry products (meat and eggs), dairy products (milk and cheese), and non-food products such as fibre (wool, mohair, cashmere, leather, and the like). Animal-derived products may also include products that can be made using animal products (e.g., fat) such as soap, creams, and such. In one example, the animal-derived product is meat, eggs, blood, brain, sperm, milk and any other tissue or sample that provides genomic DNA. In particular, the animal-derived product is meat. In one example, the animal-derived product sample may be a single type of meat, different types of meat, a single part of a type of meat, different parts of a single type of meat or different parts of different types of meat. In the event the animal is an aquatic animal, these products from animals may include meat and meat products, also including eggs, fat, flesh, blood, processed meat and lesser-known products, and non-food products such as fibre (shells, scales and the like). Animal-derived products may also include products that can be made using animal products (e.g. fish oil) such as tablets, powder and such. In one example, the animal-derived product is meat, eggs, blood, brain, shell, scale, skin, tissue, abdominal muscle tissue or any other tissue or sample that provides genomic DNA. In particular, the animal-derived product is meat, skin, blood, trimmings or any organ from the aquatic animal. In particular, trimmings are used as biproducts for fish meal/oil which end up in the animal feed industry or pets. The sample may be from any biological entity having a DNA genome and DNA genome methylation. In particular, the methylation site is a CpG site.

The term “test” used in conjunction with the term animal herein refers to an animal that is introduced to the array according to any aspect of the present invention and is the basis for an analysis application of the present invention. An “(individual) test subject”, an “(individual) group of test subjects” or a “test profile” or an ‘test animal derived product’ is therefore a (individual) subject or group of subjects being tested according to the invention or a profile being obtained or generated in this context. Similarly, the term ‘sample’ and/or ‘test animal-derived product sample’ used in accordance with any aspect of the present invention refers to an entity that may be subject to the method of the present invention. Conversely, the term “reference” or ‘control’ shall denote, mostly predetermined, entities which are used for a comparison with the test entity. In particular, a sample may be any (test) animal-derived product that may be subject to the method of the present invention to determine any feature of the animal (i.e., biological age, geographical origin, rearing method etc.) by first determining the DNA methylation profile and then comparing this test methylation profile with a control and a ‘control’ refers to an animal where the features as mentioned above are already known and where the methylation status is already known and used as a reference.

The term ‘contacting’, as used herein, means bringing about direct contact between the genomic material sample and DNA methylation-based array. For example, the genomic material sample may be DNA that is extracted from the biological sample from the test animal, and this directly brought into contact with the probe in the DNA methylation-based array.

The monogastric livestock according to any aspect of the present invention includes terrestrial and aquatic livestock with only a single compartment stomach. In particular, livestock may be rearing animals selected from terrestrial and aquatic livestock. Even more in particular, monogastric livestock excludes animals with compartmentalised stomachs known as ruminants which includes goats, sheep, cows, bison etc. In particular, monogastric terrestrial livestock may include pigs, horses, donkeys, mules, rabbits, chickens, turkeys and other gallinaceous birds, ducks, geese, quail, and the like. The term monogastric terrestrial livestock refers to the same animals as monogastric farm animals.

As used herein, the term “aquatic monogastric livestock” refers to any organism that is reared entirely in water or that lives predominantly in water, especially compared with terrestrial animals that have a single compartment stomach. These aquatic monogastric livestock may live in different water forms, such as seas, oceans, rivers, lakes, ponds, etc. More in particular, the aquatic monogastric livestock according to any aspect of the present invention may be may any fish, cephalopod, aquatic molluscs, or aquatic crustaceans, at all life stages, including eggs, sperm and gametes. Even more in particular, the ‘aquatic monogastric livestock means animals of the following species: (i) fish belonging to the superclass Agnatha and to the classes Chondrichthyes, Sarcopterygii and Actinopterygii; and (ii) aquatic crustaceans belonging to the subphylum Crustacea. Even more in particular, the aquatic monogastric livestock according to any aspect of the present invention may be aquatic livestock used in aquaculture. Some non-limiting examples of aquatic monogastric livestock according to any aspect of the present invention include vertebrate fish such as barramundi, carp, catfish, halibut, marbled crayfish, marine and brackish fishes, pangasius, rainbow trout, salmonids, sea bass, sea bream, tilapia, and turbot. The other monogastric livestock may also include marine shrimp, mitten crabs, mussels, oysters, scallops, soft-shelled crabs, soft-shelled turtles, tiger prawns, white-leg prawn, shrimp, octopus, squid and other decapod crustaceans, bivalves and gastropods. In another example, the test animal used in the method according to any aspect of the present invention may be monogastric livestock (terrestrial and aquatic) and crustaceans, bivalves and gastropods.

According to another aspect of the present invention, there is provided a method for the identification of the geographic origin of a test animal-derived product, the method comprising the steps of:
(a) carrying out the method according to any aspect of the present invention, to determine a test methylation profile of the test animal-derived product; and
(b) comparing the test methylation profile determined in (a) with one or more predetermined reference methylation profiles, wherein each of the one or more predetermined reference methylation profiles is specific for a distinct geographic origin of subjects which are of the same biological taxon as the test animal;
   wherein if the test methylation profile is significantly similar to one of the predetermined reference methylation profiles, the test animal-derived product has a geographical origin similar to the subjects of the predetermined reference methylation profile; and/or wherein if the test methylation profile is different to one of the predetermined reference methylation profiles, the test animal-derived product has a geographical origin different to the subjects of the predetermined reference methylation profile and wherein the test animal is a monogastric livestock.

The term “geographic origin” used herein relates to a geographic location which is distinguished from other geographic locations by one or more environmental parameters of the test animal. Such environmental parameters depend on the habitat of the animal and may be different in case the animal lives or is cultured in water, on or in soil, or may be selected from a food or air parameter etc. In one example, for sweet water crabs (such as the marbled crayfish), relevant environmental parameters may be selected from pH, water hardness, manganese content, iron content, and aluminum content. However, environmental parameters that are relevant may vary greatly depending on the taxon or species of the animal. Similarly, a habitat for an animal that lives in water may also vary for example, these habitats can be selected from standing or flowing waters such as lakes, rivers, aqua farms, other pools or bodies of water or ponds. A geographic origin shall be understood to be a geographic location that is considered to be the habitat, where the test animal, was birthed, hatched and/or reared, or at least reared for a significant time during their lifetime.

As used herein, the term “pre-selected methylation sites” refers to methylation sites that were selected from genes or regions that showed the highest degree of methylation variation during the training of the method and fulfils certain quality criteria such as a minimum sequencing coverage of ≥5× were considered and for ≥5 qualified CpG sites. Additionally, genes that have an average methylation level <0.1 or an average methylation level >0.9 can be excluded due to their limited dynamic range. “Reference methylation profiles” may be defined on the basis of multiple training samples using multivariate statistical methods, such as such as Principal Component analysis or Multi-Dimensional Scaling.

The term “significantly similar” as used herein, and in particular in context with the comparison of methylation profiles (such as the comparison between test profiles (from test subject(s) and reference profiles) shall mean a similarity observed by statistical means (i.e. by using bioinformatics) and/or also by observation using the eye. A significant similarity is observed for example if a test profile overlaps with a reference profile that is defined by multiple training samples through multivariate statistical methods, such as Principal Component analysis or Multi-Dimensional Scaling. In particular, a test profile is significantly similar to the pre-determined reference profile if more than 50, 55, 60, 65, 70, 75, 80, 85, 90, 95% of the methylation pattern/profile overlaps with that of the reference profile. A similarity of a test profile to more than one, such as two, three or even all reference profiles reduce the significance of the similarity.

The term “pre-determined reference profile” as used herein refers to a typical or standard methylation profile of the genomic material of a living organism with a specific feature dependent on the context where the term is used. In one example, for a method for the identification of the geographic origin of a test animal-derived product according to any aspect of the present invention, the term “pre-determined reference profile” refers to a typical or standard methylation profile of the genomic material of a living organism of a specific geographical origin. The pre-determined reference profile may be obtained from a control subject. For example, the control subject may a living organism of the same species as the test subject which has a known geographical origin. Alternatively, the pre-determined reference profile may be obtained from a variety of organisms living in the specific geographical origin. The methylation profile of different organisms of a specific geographical origin may be identical. There may be a compilation of several pre-determined reference profiles and comparing the methylation profile of the test subject with the pre-determined reference profiles in the compilation may enable identifying the specific pre-determined reference profile that is similar to the methylation profile of the test subject and then the geographical origin of the test subject may be deduced to be that of the pre-determined reference profile.

The term “similar” used in relation to the geographical origin refers to the habitat or geographical origin of the test subject(s) based on the habitat or geographical origin of the organism from which the pre-determined reference profile was obtained. The term ‘similar’ may refer to the type of habitat, the environmental parameters of the habitat, the country where the habitat is located and the like. The geographical origin of the test subject may be 50, 55, 60, 65, 70, 75, 80, 85, 90, 95% similar to that of the geographical origin of the pre-determined reference profile based on at least one or more environmental parameters as defined above under ‘geographical origin’.

A further disclosure of the technicalities of the connection between DNA methylation changes and the use of these to determine geographical origins is disclosed in WO2022/023208.

According to another aspect of the present invention, there is provided an in vitro method for predicting the biological age of a test animal from which a product is derived, the method comprising the step of:
(a) carrying out the method according to any aspect of the present invention, to determine a test methylation profile of the test animal; and
(b) comparing the test methylation profile from (a) with the methylation profile from an age-correlated reference sample, thereby establishing the epigenetic age and predicting the biological age of the test animal from which the product is derived; and
   wherein the test animal is a monogastric livestock.

The age-correlated reference sample serves as a control and represents an average methylation level at a pre-determined and specific chronological age.

The term “chronological age” refers to the calendar time that has passed from birth/hatch.

The epigenetic age depends on the biological state or condition of an individual or of a population and takes into account the circumstances of life (such as stress, nutrition, etc.). The terms “epigenetic age”, “methylation age”, and “biological age” have identical meanings and are used interchangeably.

Epigenetic age may match or mismatch with chronological age. Deviations of the epigenetic age from the chronological age are age acceleration or age deceleration. Accordingly, epigenetic age may also be determined by comparison of the methylation levels of the methylation markers (i.e. CpG sites) in the genomic DNA from the sample to be tested with the methylation status of the same markers (i.e. CpG sites) from an age-correlated reference sample.

In one example, for an in vitro method for predicting the biological age of a test animal from which a product is derived according to any aspect of the present invention, the term “pre-determined reference profile” refers to a typical or standard methylation profile of the genomic material of a living organism of a known chronical age. The pre-determined reference profile may be obtained from a control subject or population of control subjects where the age of the animals is known. There may be a compilation of several pre-determined reference profiles and comparing the methylation profile of the test subject with the pre-determined reference profiles in the compilation may enable identifying the specific pre-determined reference profile that is similar to the methylation profile of the test subject and then the biological age of the test subject may be deduced to be that of the pre-determined reference profile.

A further disclosure of the technicalities of the connection between DNA methylation changes and the use of these changes to determine biological age of an animal in question is provided at least in WO 2021/148593 and WO 2021/148601.

The method according to this aspect of the present invention may also be used to determine the health status of a (test) animal. In particular, the health status may refer to whether an animal has a disease, the animal welfare of the animal based on the environment and/or food safety of the animal. Methylation profiles for each of these components may be developed to create a panel of reference methylation profiles that may then be used to determine the health status of the test animal.

According to a further aspect of the present invention, there is provided a method of determining if a test animal from which a product is derived has been treated and/or is currently undergoing treatment with at least one antibiotic and/or veterinary chemical, the method comprising:
(a) carrying out the method according to any aspect of the present invention, to determine a test methylation profile of the test animal; and
(b) comparing the test methylation profile obtained from (a) with a reference methylation profile obtained from a control animal or a control animal-derived product, where the control animal was not treated and/or is not currently undergoing treatment with at least one antibiotic and/or veterinary chemical,
   wherein a difference in the test methylation profile of (a) compared to the reference methylation profile from the control animal, is indicative of the test animal having been treated and/or is currently undergoing treatment with at least one antibiotic and/or veterinary chemical; and/or wherein a significant similarity in the test methylation profile of (a) compared to the reference methylation profile, is indicative of the test animal having not been treated and/or is currently not undergoing treatment with at least one antibiotic and/or veterinary chemical; and
   wherein the test animal is a monogastric livestock.

As used herein the term ‘antibiotic’ refers to any medicine that may be fed to the terrestrial animal for therapeutic and/or preventive purposes. The antibiotic may be administered by any method known in the art. The antibiotic may be fed orally to the terrestrial animal according to any aspect of the present invention in the animal feed, or water such that it is ingested. In another example, the antibiotic may be injected into the animal. In one example, the antibiotic may be introduced into the terrestrial animal via udder injections. A skilled person would understand the best way to provide the antibiotic to the animal based on the specific biological taxon of the animal, the type of antibiotic and the disease to be treated or prevented. In particular, the antibiotic according to any aspect of the present invention may be selected from the group of classes consisting of amphenicols, aminocyclitols, aminoglycosides, ansamycins, beta-lactams, carbaephem, carbapenems, cephalosporins, chloramphenicol, fluoroquinolones, glycopeptides, glycylcyclines, ketolides, lincosamides, lipopeptides, macrolides, monobactams, nitrofurans, nitroimidazoles, oxazolidinones, penicillins, phosphonic acid derivatives, pleuromutilins, polymyxins, polypeptides, quinolones, rifamycins, riminofenazines, steroid antibacterials, streptogramins, sulfonamides, tetracyclines, and trimethoprim. More in particular, the antibiotic may be selected from the group consisting of tetracycline hydrochloride, Amoxicillin and Colist.

The test animal according to any aspect of the present invention may be fed with at least one or more antibiotics mentioned above simultaneously or consecutively. The contact of antibiotics with the terrestrial animal may bring about epigenetic changes, at least DNA methylation changes, that may then be determined using the method according to any aspect of the present invention. The concentration of antibiotics in each dose and/or the period of time the antibiotic has been given to the test animal may affect the extend of differential methylation in the test animal relative to the control animal. It is within the knowledge of a skilled person to determine the concentration of each dose and the period of antibiotic exposure that the test animal requires depending on whether the antibiotic is given for preventive or therapeutic measures.

As used herein the term ‘veterinary chemical’ refers to drugs or medicines used to treat or prevent disease, injury, and pests in animals. In particular, ‘veterinary chemical’ may refer to an anti-parasitic, an anti-viral, a feed additive, a water additive, a disinfectant, glutaraldehyde, formalin, mixtures thereof and the like. In one example, the feed additive may be a coccidiostat or ionophore. Water additive refers to chemicals that may be added to water lines rather than into feed of the terrestrial animals. The veterinary chemical may be administered by any method known in the art to the terrestrial animal.

The test animal used in the method according to any aspect of the present invention may be brought into contact with both an antibiotic and a veterinary chemical simultaneously and/or consequently. The change in the internal environment of the test animal leads to an epigenetic change and this can be determined using the method according to any aspect of the present invention.

In particular, in the method according to any aspect of the present invention, in step (a) the methylation status of at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 CpG sites are determined. A skilled person would be capable of determining the number of CpG sites that need to be used in step (a) according to any aspect of the present invention. Even more in particular, the methylation status of at least two CpG sites are determined in step (a) of the method according to any aspect of the present invention.

According to yet another aspect of the present invention, there is provided a method of determining if a test animal from which a product is derived has been treated and/or is currently undergoing treatment with at least one antibiotic, and if so, determining the distinct class of antibiotics with which the test animal is being treated and/or is currently undergoing treatment, the method comprising:
(a) carrying out the method according to any aspect of the present invention, to determine a test methylation profile of the test animal; and
(b) comparing the test methylation profile obtained from (a) with one or more predetermined reference methylation profiles, wherein each of the predetermined reference methylation profiles is from a different animal of the same biological taxon as the test animal, and each of the different animals was treated with a different class of antibiotics,
   wherein if the test methylation profile of (a) is significantly similar to one of the predetermined reference methylation profiles, the test animal from which the product is derived is confirmed for being treated with the same class of antibiotics with which the animal with the predetermined reference profile is treated with and the test animal has been confirmed for having been treated and/or is currently undergoing treatment with the distinct class of antibiotics; and/or
   wherein if the test methylation profile of (a) is significantly different to one of the predetermined reference methylation profiles, the test animal from which the product is derived is confirmed for not being treated with the same class of antibiotics with which the animal with the predetermined reference profile is treated with and the test animal has not been confirmed for having been treated and/or is currently undergoing treatment with the distinct class of antibiotics; and
   wherein the test animal is a monogastric livestock.

In particular, the distinct classes of antibiotics are amphenicols, aminocyclitols, aminoglycosides, ansamycins, beta-lactams, carbaephem, carbapenems, cephalosporins, chloramphenicol, fluoroquinolones, glycopeptides, glycylcyclines, ketolides, lincosamides, lipopeptides, macrolides, monobactams, nitrofurans, nitroimidazoles, oxazolidinones, penicillins, phosphorus containing antibiotics; and/or the veterinary chemical is an anti-parasitic, an anti-viral, a feed additive, a disinfectant, glutaraldehyde, and/or formalin.

In one example, a panel of pre-determined reference profiles may be prepared for different animals to be used as a control where each animal has been contacted with a different class of antibiotics and/or each part of the animal (i.e., tissue, muscle, blood, skin) has its own unique pre-determined methylation reference profile that also forms a part of the panel of pre-determined reference profiles. Different animals from the same biological taxon as the test animal, each being treated with a different class of antibiotic may have its own panel of pre-determined reference profiles for each part of the animal or animal-derived product that is used as the genomic material. For example, each panel may be specific for a single animal in contact with a first antibiotic and/or veterinary chemical where each reference profile may be distinct for a part of the animal from which the genomic material is extracted. There will thus be a compilation of panels of pre-determined reference profiles, each panel specific for one control animal of the same biological taxon as the test animal, the control animal being in contact with or is in contact with a first, second, third and the like antibiotic and/or veterinary chemical. When a test methylation profile from an unknown animal-derived product sample is obtained, this is then compared with the different panels of pre-determined reference profiles for the same animal taxon as the test animal to determine the distinct class of antibiotic and/or veterinary chemical the test animal is or was in contact with.

According to yet another aspect of the present invention refers to a method of determining if a test animal and/or a test animal from which a product is derived has been treated and/or is currently undergoing treatment with at least one antibiotic, and if so, determining if the antibiotic is used as a growth promotant or as a therapeutant, the method comprising:
(a) carrying out the method according to any aspect of the present invention, to determine a test methylation profile of the test animal; and
(b) comparing the test methylation profile obtained from (a) with one or more predetermined reference methylation profiles, wherein each of the predetermined reference methylation profiles is from a different control animal of the same biological taxon as the test animal, and each of the different control animals was treated with the antibiotic as a growth promotant or therapeutant,
   wherein if the test methylation profile of (a) is significantly similar to one of the predetermined reference methylation profiles, the test animal is confirmed for being treated with the antibiotic in the same way that the control animal with the similar predetermined reference profile is treated and the test animal has been confirmed for having been treated and/or is currently undergoing treatment with the antibiotic as a growth promotant or therapeutant.

As used herein the term ‘growth promotant’ refers to the antibiotic being used to help increase the efficiency of animal production by increasing weight gain and product output. The antibiotic may be used as a growth promotant in contrast to it being used as a therapeutant (i.e., for treatment of a disease)

According to a further aspect of the present invention, there is provided a method of determining if a test animal from which a product is derived underwent a withdrawal period of no treatment with at least one antibiotic and/or veterinary chemical prior to the product being obtained, the method comprising:
(a) carrying out the method according to any aspect of the present invention, to determine a test methylation profile of the test animal; and
(b) comparing the test methylation profile obtained from (a) with at least two predetermined reference methylation profiles, wherein at least one of the predetermined reference methylation profiles is from a control animal that underwent a withdrawal period and at the other one of the predetermined reference methylation profiles is from a control animal of the same biological taxon as the test animal, that did not undergo a withdrawal period before the product was obtained,
   wherein if the test methylation profile of (a) is significantly similar to the predetermined reference methylation profile, the test animal is confirmed for having undergone a withdrawal period or not.

As used herein, the term ‘withdrawal period’ refers to the period from the time point where the animal is no longer fed the antibiotic and/or veterinary chemical to the point where the remaining antibiotic is broken down in the body until it becomes a non-functional agent and is finally, eliminated from the body of the animal. Withdrawal periods of different antibiotics may vary from 1 or 2 days to couple of weeks. A “withdrawal” period is required from the time antibiotics are administered until it is legal to slaughter the animal or to derive products from the animal. The time it therefore takes the body to break down the antibiotic until it is no longer functional, or present is called the withdrawal time (or withdrawal period). Once the withdrawal period has passed the antibiotic has been eliminated from the animal's system.

According to a further aspect of the present invention, there is provided a method of certifying a test animal-derived product sample, the method comprising the steps of:
(a) carrying out the method according to any aspect of the present invention, to determine a test methylation profile of the test animal; and
(b) comparing the test methylation profile obtained from (a) with a reference methylation profile obtained from a control animal of the same biological taxon of the test animal from which the product sample is derived from, where the control animal is not slaughtered by a single cut across the throat severing both carotid arteries, both jugular veins, both vagus nerves, trachea and/or esophagus and/or the control animal is not bled to death
   wherein a difference in the test methylation profile of (a) compared to the reference methylation profile from the control animal, is indicative of the test animal having been slaughtered by a single cut across the throat severing both carotid arteries, both jugular veins, both vagus nerves, trachea and/or esophagus and/or of the test animal having been bled to death and the test animal-derived product is certified so; and/or
   wherein a significant similarity e in the test methylation profile of (a) compared to the reference methylation profile from the control animal, is indicative of the test animal not having been slaughtered by a single cut across the throat severing both carotid arteries, both jugular veins, both vagus nerves, trachea and/or esophagus and/or of the test animal having been bled to death and the test animal-derived product is certified so; and wherein the test animal is a monogastric livestock.

The term ‘certification of quality’ refers to a certificate or a confirmation given by designated certification agencies that endorse the quality of a particular animal derived product, including food for use and/or consumption by human beings. The term ‘certification of quality’ is used interchangeably with the term ‘certification’. These certifications are usually found on the packaging of the animal-derived product including food to be consumed and are printed by the manufactures of the products. ‘Haltungsform’, ‘Tierwohl’, ‘Ohne Gentechnik’, ‘halal’, ‘kosher’, ‘organic’, free range’, ‘pasture raised’, ‘grass fed’, ‘grain fed’, ‘vegetarian’, ‘raised without hormones’, and the like. There are different certifications based on the country as well. For example, like Haltungsform in Germany, other certifications include Red Tractor (UK), Label Rouge (France), USDA Grade (USA) etc., and other safe labels that confirm that a product sold has been prepared in accordance with specific religious or safety regulations. Specifically, the term ‘certification of food quality’ refers to a certificate or a confirmation given by designated certification agencies that endorse the quality, source or means of slaughter of a particular food for consumption by human beings. According to any aspect of the present invention, the certified quality may be a distinct certified food quality, or distinct certification and this may be kosher, non-kosher, halal or non-halal.

In one example, the distinct certification or certification of the sample X according to any aspect of the present invention may be kosher, non-kosher, halal or non-halal. More in particular, kosher or halal refers to the sample X originating from an animal that was slaughtered by a single cut across the throat severing both carotid arteries, both jugular veins, both vagus nerves, the trachea and/or the esophagus. Even more in particular, the animal is drained of blood.

The term ‘kosher’ used in combination with food according to any aspect of the present invention refers to food that conforms to Jewish dietary regulations of kashrut (dietary law) or food that may be consumed according to halakha (law). Kosher used in relation to meat relates particularly to a manner in which animals are prepared for consumption. According to Jewish tradition, meat may be considered kosher when the meat comes from animals that have been slaughtered according to Jewish law where the animal is killed by a single cut across the throat to a precise depth, severing both carotid arteries, both jugular veins, both vagus nerves, the trachea and the esophagus, no higher than the epiglottis and no lower than where cilia begin inside the trachea, causing the animal to bleed to death. Such slaughter is to be carried out using a large, razor-sharp knife, which is checked before each slaughter to ensure that it has no irregularities (such as nicks and dents). The slaughter is usually also carried out by a shochet or a rabbi. Kosher meat usually refers to most meats excluding pig. In particular, kosher meat may be selected from beef, chicken, lamb, mutton, goat meat and mixtures thereof. Kosher meat does not include shellfish, which under Jewish tradition is not permitted for consumption. Although Jewish traditions permit consumption of vertebrate fish, since there is no special method of slaughtering vertebrate fish, all vertebrate fish may be considered kosher. Any food or meat that does not fall within the definition of ‘kosher’ will then be considered as ‘non-kosher’.

The term ‘halal’ used in combination with food according to any aspect of the present invention refers to food that conforms to Islamic dietary laws and especially meat processed and prepared in accordance with those requirements. Similar to the way kosher meat is prepared, in Islamic tradition, animals are slaughtered according to Dhabīhah where the animal is slaughtered using a cut across the neck with a non-serrated sharp blade in a single clean attempt to make an incision that cuts the front of the throat, oesophagus and jugular veins but not the spinal cord. In addition to the direction, permitted animals should be slaughtered upon utterance of the Islamic prayer Bismillah. The animal must also be drained of blood after the slaughter. The slaughter must be performed by an adult Muslim. Halal meat usually refers to most meats excluding pig. In particular, halal meat may be selected from beef, chicken, lamb, mutton, goat meat and mixtures thereof. Although Islamic traditions permit consumption of shellfish and vertebrate fish, since there is no special method of preparing shellfish, all shellfish and vertebrate fish may be considered halal. Any food or meat that does not fall within the definition of ‘halal’ will then be considered as ‘non-halal. The definition of halal is further provided in https://www.smiic.org/en/project/24 (Organisation of Islamic Cooperation (OIC)/Standards and Metrology Institute for the Islamic Countries (SMIIC), OIC/SMIIC 1:2019 General Requirements for Halal Food. accessed on 8 Jun. 2022).

The term “pre-determined reference profile” as used herein refers to a typical or standard methylation profile of the genomic material of a type of reference animal-derived product that is confirmed to be correctly labelled or certified. In one example, the pre-determined reference profile may be used in the context of a control animal, where the control animal has been correctly certified (i.e. where the control animal has neither been slaughtered by a single cut across the throat severing both carotid arteries, both jugular veins, both vagus nerves, trachea and/or esophagus nor of having been bled to death). The control animal may thus have been slaughtered using non-halal and non-kosher means of slaughtering that may be termed the normal means of slaughtering. A panel of pre-determined reference profiles for control animals may include profiles from different samples that have been obtained from different parts of control animals (not slaughtered by a single cut across the throat severing both carotid arteries, both jugular veins, both vagus nerves, trachea and/or esophagus nor of having been bled to death). For example, the panel of pre-determined reference profiles may include at least one profile for egg, at least one profile for meat (muscle, tissue, organs etc.), at least one profile for milk and the like. Each of these samples may have its own unique pre-determined methylation reference profile that also forms a part of the panel of pre-determined reference profiles.

In another example, the pre-determined reference profile may be used in the context of reference animals that may have been slaughtered by a single cut across the throat severing both carotid arteries, both jugular veins, both vagus nerves, trachea and/or esophagus and/or having been bled to death. A panel of pre-determined reference profiles may be prepared for different samples that are from animals that have been confirmed to be slaughtered by a single cut across the throat severing both carotid arteries, both jugular veins, both vagus nerves, trachea and/or esophagus and/or having been bled to death. Again here, there may be a panel of pre-determined reference profiles for each product that is derived from the animal that has been slaughtered by a single cut across the throat severing both carotid arteries, both jugular veins, both vagus nerves, trachea and/or esophagus and/or having been bled to death. For example, the panel of pre-determined reference profiles may include at least one profile for egg, at least one profile for meat (muscle, tissue, organs etc.), at least one profile for milk and the like from animals that have been slaughtered by a single cut across the throat severing both carotid arteries, both jugular veins, both vagus nerves, trachea and/or esophagus and/or having been bled to death. Each of these samples may have its own unique pre-determined methylation reference profile that also forms a part of the panel of pre-determined reference profiles.

For example, the control may a type of meat of the same species or the same animal taxon as the animal-derived product sample or unknown sample which has a known certification. Alternatively, the pre-determined reference profile may be obtained from different types of meat with a known certification. In one example, the control may be chicken meat that is certified ‘halal’. The halal chicken meat may be a mix of different parts of the chicken (i.e. breast, thigh, kidney, liver etc.). In another example, the halal chicken meat may be from one part of a chicken. From the halal chicken meat, a methylation profile may be obtained to be the pre-determined reference profile (the control) that may be used to determine if the test sample (meat) is halal or non-halal by determining if the methylation profile of the test sample is significantly similar to the pre-determined reference profile. The methylation profile of different types of halal meat from one species of animal may be identical. The methylation profile of different types of halal meat from different species of animals may also be identical. In one example, the control may be chicken meat that is certified ‘kosher’. The kosher chicken meat may be a mix of different parts of the chicken (i.e. breast, thigh, kidney, liver etc.). In another example, the kosher chicken meat may be from one part of a chicken. From the kosher chicken meat, a methylation profile may be obtained to be the pre-determined reference profile (the control) that may be used to determine if the test sample (meat) is kosher or non-kosher by determining if the methylation profile of the test sample is significantly similar to the pre-determined reference profile. The methylation profile of different types of kosher meat from one species of animal may be identical. The methylation profile of different types of kosher meat from different species of animals may also be identical.

There may be a compilation of several pre-determined reference profiles and comparing the methylation profile of the test sample with the pre-determined reference profiles in the compilation may enable identifying the specific pre-determined reference profile that is (significantly) similar to the methylation profile of the test sample and then the test sample may be confirmed to be slaughtered by a single cut across the throat severing both carotid arteries, both jugular veins, both vagus nerves, trachea and/or esophagus and/or having been bled to death or not. In one example, the pre-determined reference profiles may include methylation profiles of different parts of meat (i.e. breast, thigh, kidney, liver, shoulder, ribs, intestines, etc.) from an animal (chicken, goat, cow, lamb, sheep etc.) that has been slaughtered by a single cut across the throat severing both carotid arteries, both jugular veins, both vagus nerves, trachea and/or esophagus and/or having been bled to death.

In particular, the panel of predetermined reference methylation profiles according to any aspect of the present invention is distinct for different test animal-derived products. That is to say, each predetermined reference methylation profile is distinct for a single animal-derived product. The panel of predetermined reference methylation profiles may thus include many different predetermined reference methylation profiles from different parts of an animal or several animals of the same biological taxon as the test animal. There will also be different panels of predetermined reference methylation profiles for different animal taxon, and the relevant panel of predetermined reference methylation profiles unique for an animal taxon will depend on the animal taxon of the test animal.

According to yet a further aspect of the present invention, there is provided a method of certifying a test animal-derived product sample, the method comprising the steps of:
(a) carrying out the method according to any aspect of the present invention, to determine a test methylation profile of the test animal; and
(b) comparing the test methylation profile obtained from (a) with a reference methylation profile obtained from a control animal of the same biological taxon of the test animal from which the product sample is derived from, where the control animal is bred under a distinct type of animal husbandry;
   wherein a significant similarity in the test methylation profile of (a) compared to the reference methylation profile from the control animal, is indicative of the test animal having been bred under the same distinct type of animal husbandry as the control animal; and/or
   wherein a difference in the test methylation profile of (a) compared to the reference methylation profile of the control animal, is indicative of the test animal having been bred under another distinct type of animal husbandry as the control animal; and wherein the test animal is a monogastric livestock.

In one example, the distinct certification or certification of sample X may be based on a type of animal husbandry that the test animal was reared under. In Germany, this is labelled as ‘Haltungsform’. There are at least four types/conditions under which the animals may be reared. These four levels of animal husbandry include Stable housing (Stallhaltung), Stable housing Plus (StallhaltungPlus), Outside climate (Außenklima) and Premium (Premium), these are also known as Haltungsform 1, 2, 3 and 4 respectively. Animal products derived from animals bred under different animal husbandry conditions may result in a different DNA methylation profile. The distinct type of animal husbandry may vary depending on the country where the method according to any aspect of the present invention is carried out. Regardless of different terminology used in different countries to describe different distinct animal husbandry practices, the overall concept of the method according to any aspect of the present invention is the same and applicable in any one of these countries.

For example, in Germany, the different distinct types of animal husbandry techniques practiced on livestock and poultry may be labelled ‘Haltungsform’ and as mentioned above, are officially and accepted by the industry to be divided into least four types/conditions under which the animals may be reared. These four levels of animal husbandry include Stable housing (Stallhaltung), Stable housing Plus (StallhaltungPlus), Outside climate (Außenklima) and Premium (Premium). Similarly, in France the livestock and poultry may be labelled ‘label rouge’, ‘organic’, or with other pictograms that display the farming methods the animal went through before the animal derived product was obtained. In the United Kingdom livestock and poultry the Red Tractor Food Assurance certification scheme exists which includes at least three levels of animal husbandry including Certified Standards, Enhanced Welfare and Free Range. Other labels existing in the United Kingdom include RSPCA Assured which certify specific animal welfare standards and several organic meat certifying schemes such as the Organic Farmers and Growers Certification and the Soil Association Organic Standard. Examples of meat certification in the United States of America (USA) includes those provided by the United States Department of Agriculture (USDA), which include Grade A Carcass Quality and Organic certifications as examples. The USDA also approves some third-party certification schemes such as provided by the nonprofit A Greener World, which include Certified Animal Welfare Approved defining husbandry related to animal welfare and Certified Grassfed defining specific feed types in animal husbandry.

The term “pre-determined reference profile” in this example may be used in the context of a control animal, where the control animal has been correctly certified (i.e. based on the animal husbandry technique under which the control animal was bred or reared. The control animal may thus have been reared under Stable housing (Stallhaltung), Stable housing Plus (StallhaltungPlus), Outside climate (Außenklima) or Premium (Premium) conditions. A panel of pre-determined reference profiles for control animals may also include profiles from different samples that have been obtained from different parts of control animals (animals reared from at least one, two, three or four of the animal husbandry conditions). For example, the panel of pre-determined reference profiles may include at least one profile for egg, at least one profile for meat (muscle, tissue, organs etc.), at least one profile for milk and the like. Each of these samples may have its own unique pre-determined methylation reference profile that also forms a part of the panel of pre-determined reference profiles. The panel may also include a pre-determined reference profile for each of these animal derived products specific for each of the four animal husbandry techniques.

A panel of pre-determined reference profiles may be prepared for different samples that are from animals that have been confirmed to be reared according to at least one of these four animal husbandry techniques. Again here, there may be a panel of pre-determined reference profiles for each product that is derived from the animal that has been bred according to at least one of these four animal husbandry techniques. For example, the panel of pre-determined reference profiles may include at least one profile for egg, at least one profile for meat (muscle, tissue, organs etc.), at least one profile for milk and the like from animals that have been bred according to the animal husbandry that falls under Stable housing (Stallhaltung). Each of these samples may have its own unique pre-determined methylation reference profile that also forms a part of the panel of pre-determined reference profiles. A second panel of pre-determined reference profiles may include at least one profile for egg, at least one profile for meat (muscle, tissue, organs etc.), at least one profile for milk and the like from animals that have been bred according to the animal husbandry that falls under Stable housing Plus (StallhaltungPlus). A third panel of pre-determined reference profiles may include at least one profile for egg, at least one profile for meat (muscle, tissue, organs etc.), at least one profile for milk and the like from animals that have been bred according to the animal husbandry that falls under Outside climate (Außenklima). A fourth panel of pre-determined reference profiles may include at least one profile for egg, at least one profile for meat (muscle, tissue, organs etc.), at least one profile for milk and the like from animals that have been bred according to the animal husbandry that falls under Premium (Premium) conditions. In one example, the panel of pre-determined reference profiles may include all four different panels. In yet another example, the panel may be based on the different animal husbandry techniques found in a particular, land, state or geographical location. The number of panels of pre-determined reference profiles may vary depending on where the method is carried out and what the animal farming and/or animal husbandry techniques practiced in the country or region may be.

The methylation profile of different types of meat from animals grown under a particular animal husbandry technique may be identical. The methylation profile of different types of meat from different species of animals reared under particular animal husbandry technique may also be identical.

There may be a compilation of several pre-determined reference profiles and comparing the methylation profile of the test sample with the pre-determined reference profiles in the compilation may enable identifying the specific pre-determined reference profile that is (significantly) similar to the methylation profile of the test sample and then the test sample may be confirmed to have been reared under a distinct animal husbandry technique or not. In one example, the pre-determined reference profiles may include methylation profiles of different parts of meat (i.e. breast, thigh, kidney, liver, shoulder, ribs, intestines, etc.) from an animal (chicken, goat, cow, lamb, sheep etc.) that has been reared under a distinct animal husbandry technique.

In particular, the panel of predetermined reference methylation profiles according to any aspect of the present invention is distinct for different test animal-derived products. That is to say, each predetermined reference methylation profile is distinct for a single animal-derived product. The panel of predetermined reference methylation profiles may thus include many different predetermined reference methylation profiles from different parts of an animal or several animals of the same biological taxon as the test animal. There will also be different panels of predetermined reference methylation profiles for different animal taxon, and the relevant panel of predetermined reference methylation profiles unique for an animal taxon will depend on the animal taxon of the test animal.

According to another aspect of the present invention, there is provided a method of certifying a test animal-derived product sample, the method comprising the steps of:
(a) carrying out the method according to any aspect of the present invention, to determine a test methylation profile of the test animal; and
(b) comparing the test methylation profile obtained from (a) with a reference methylation profile obtained from a control animal of the same biological taxon of the test animal from which the product sample is derived from, where the control animal is reared using conventional farming techniques;
   wherein a difference in the test methylation profile of (a) compared to the reference methylation profile from the control animal, is indicative of the test animal having been reared according to organic standards and the test animal-derived product is certified so.

According to one aspect of the present invention, there is provided a method for the determination of whether a test animal-derived product is farmed or wild caught, the method comprising the steps of:
(a) carrying out the method according to any aspect of the present invention, to determine a test methylation profile of the test animal-derived product; and
(b) comparing the test methylation profile determined in (a) with one or more predetermined reference methylation profiles, wherein each of the one or more predetermined reference methylation profiles is specific for wild caught or farmed subjects which are of the same biological taxon as the test animal;
   wherein if the test methylation profile is significantly similar to one of the predetermined reference methylation profiles, the test animal-derived product is either wild caught or farmed.

According to a further aspect of the present invention, there is provided a method of determining the supplier from which a test animal-derived product sample originates, the method comprising the steps of:
(a) carrying out the method according to any aspect of the present invention, to determine a test methylation profile of the test animal-derived product; and
(b) comparing the test methylation profile determined in (a) with a panel of predetermined reference methylation profiles of the same biological taxon of the test animal from which the product sample derives, wherein each of the predetermined reference methylation profiles is from a different reference animal and/or different supplier,
   wherein if the test methylation profile of (a) is significantly similar to one of the predetermined reference methylation profiles, the test animal-derived product sample is confirmed of originating from a first supplier from which a first reference animal with the predetermined reference profile is obtained; and/or
   wherein if the test methylation profile of (a) is different to one of the predetermined reference methylation profiles, the test animal-derived product sample is confirmed of not originating from a first supplier from which a first reference animal with the predetermined reference profile is obtained; and wherein the test animal is a monogastric livestock.

The method according to this aspect of the present invention may thus enable the tracing of the original slaughterhouse, farm, produce, supplier etc. from which the test animal material originates from and thereby the test animal derived product.

The method according to this aspect of the present invention may be used to identify an unknown sample (i.e. animal-derived product sample) based on DNA methylation patterns. These DNA methylation patterns may then be compared with reference DNA methylation patterns to trace the animal-derived product sample back to the slaughterhouse or farmhouse from which the sample originates and then determine whether the unknown sample corresponds to an animal which has been slaughtered according to Kosher, Halal, non-Kosher or non-Halal practices. In this way, a buyer or a consumer of meat can verify that meat being sold as or marketed as being Kosher or Halal is genuine.

The term ‘supplier’ used herein refers to the entity that provides the animal derived product. This can be considered the slaughterhouse according to any aspect of the present invention as the slaughterhouse is where the animal is killed according to specific practices and the different animal derived products which include animal products and animal by-products originate from. The slaughterhouse or slaughter facility typically slaughters the animal and then chills, ages and cuts the carcass into the various cuts of meat and packs those cuts for shipment to distributors and retailers. The slaughterhouse is also where non-food products originate from. The supplier may be certified. A ‘certified supplier’ means a supplier that has been assessed for quality, business, technical, environmental, health and safety considerations and subsequently approved by a government or third-party agency or agencies. In one example, the supplier may be certified ‘halal’. This means that all the animal derived products that are supplied by the certified supplier will be certified ‘halal’. In another example, the supplier may be certified ‘kosher’. This means that all the animal derived products that are supplied by or originated from the certified supplier will be certified ‘kosher’. The supplier is certified kosher or halal where the animal is slaughtered by a single cut across the throat severing both carotid arteries, both jugular veins, both vagus nerves, the trachea and/or the esophagus. In addition, or alternatively, the supplier is certified kosher or halal where the animal is drained of blood. The animal may be selected from the group consisting of cow, sheep, goat, camel, chicken, goose, duck and turkey.

The term “pre-determined reference profile” used according to this aspect of the present invention may be obtained from a control subject. For example, the control may be a piece of meat from an animal that has been slaughtered by a certified supplier. A panel of pre-determined reference profiles may be prepared for each certified supplier where each pre-determined profile is unique for each animal that has been slaughtered by the certified supplier and/or each part of the animal (i.e. tissue, muscle, blood, skin) has its own unique pre-determined methylation reference profile that also forms a part of the panel of pre-determined reference profiles. Different certified suppliers may then have different panels of pre-determined reference profiles. For example, each panel may be specific for a single animal where each reference profile may be distinct for a part of the animal from which the genomic material is extracted. When a test methylation profile from an unknown animal-derived product sample is obtained, this is then compared with the different panels of pre-determined reference profiles to trace the actual animal from which the animal-derived product sample comes from. If the test methylation profile is significantly similar to one of the pre-determined reference profiles, then the origin of the animal-derived product sample can be determined, and the appropriate certification may be given to the sample, or the sample can be certified for its quality. If the test methylation profile is found to be not significantly similar to any one of the pre-determined reference profiles, then the animal-derived product sample is confirmed not to be from the list of certified suppliers and cannot be given the certification or cannot be certified for its quality.

For example, the control may a type of meat of the same species or the same animal taxon as the animal-derived product sample or unknown sample which has a known certification or certification of food quality. Alternatively, the pre-determined reference profile may be obtained from different types of meat with a known certification of food quality. In one example, the control may be chicken meat that is certified ‘halal’ from a first certified supplier. The halal chicken meat may be a mix of different parts of the chicken (i.e. breast, thigh, kidney, liver etc.) from a single chicken. In another example, the halal chicken meat may be a mix of different parts of the chicken (i.e. breast, thigh, kidney, liver etc.) from a few chickens from a first certified supplier. In yet another example, the halal chicken meat may be from one part of a single chicken from a first certified supplier. From the halal chicken meat, a methylation profile may be obtained to be the pre-determined reference profile (the control) that may be used to determine if the test sample (meat) is halal or non-halal by determining if the test sample is from the first certified supplier. The methylation profile of different types of halal meat from one species of animal may be identical. The methylation profile of different types of halal meat from different species of animals may also be identical. In another example, the control may be chicken meat that is certified ‘kosher. The kosher chicken meat may be a mix of different parts of the chicken (i.e. breast, thigh, kidney, liver etc.) from a single chicken from a second certified supplier. In another example, the kosher chicken meat may be a mix of different parts of the chicken (i.e. breast, thigh, kidney, liver etc.) from a few chickens from the second certified supplier. In yet another example, the kosher chicken meat may be from one part of a single chicken from the second certified supplier. From the kosher chicken meat, a methylation profile may be obtained to be the pre-determined reference profile (the control) that may be used to determine if the test sample (meat) is kosher or non-kosher by first determining if the test sample is from the second certified supplier. The methylation profile of different types of kosher meat from one species of animal may be identical. The methylation profile of different types of kosher meat from different species of animals may also be identical.

There may be a compilation of several pre-determined reference profiles and comparing the methylation profile of the test sample with the pre-determined reference profiles in the compilation may enable identifying the specific pre-determined reference profile that is (significantly) similar to the methylation profile of the test sample and then the test sample may be traced back to its origins or to the supplier and thereby the certification of quality of the test sample may be deduced to be that of the pre-determined reference profile. In one example, the pre-determined reference profiles may include methylation profiles of different parts of meat (i.e. breast, thigh, kidney, liver, shoulder, ribs, intestines, etc.) from a single animal (chicken, goat, cow, lamb, sheep etc.) or at least two animals of the same species or at least two animals from different species. The different unique methylation profiles are reference epigenetic signatures of meat from a specific source (EpiTrace®).

In particular, the panel of predetermined reference methylation profiles according to any aspect of the present invention is distinct for different test animal-derived products. That is to say, each predetermined reference methylation profile is distinct for a single animal-derived product from one animal from one unique supplier. The panel of predetermined reference methylation profiles may thus include many different predetermined reference methylation profiles from different parts of a single animal from the unique supplier, many different predetermined reference methylation profiles from different animals from the unique supplier and many different predetermined reference methylation profiles from different parts of different animals from different suppliers. There will also be different panels of predetermined reference methylation profiles for different animal taxon, and the relevant panel of predetermined reference methylation profiles unique for an animal taxon will depend on the animal taxon of the test animal.

In particular, the first supplier according to any aspect of the present invention is certified, and the test animal-derived product sample has the same certification as the first reference animal from which the significantly similar predetermined reference profile is obtained.

More in particular, the first supplier according to any aspect of the present invention is certified kosher or halal where the animal is slaughtered by a single cut across the throat severing both carotid arteries, both jugular veins, both vagus nerves, the trachea and/or the esophagus. In addition, or alternatively, the first supplier may be certified kosher or halal where the animal is drained of blood.

A method of determining if a test animal derived product sample is derived from an animal that is suitable for selective breeding, the method comprising the steps of:
(a) carrying out the method according to any aspect of the present invention, to determine a test methylation profile of the test animal; and
(b) comparing the test methylation profile obtained from (a) with a reference methylation profile obtained from a control animal of the same biological taxon of the test animal from which the product sample is derived from, where the control animal comprises at least one phenotype of interest for selective breeding,
   wherein a significant similarity in the test methylation profile of (a) compared to the reference methylation profile from the control animal, is indicative of the test animal being suitable for selective breeding; and/or
   wherein a difference in the test methylation profile of (a) compared to the reference methylation profile of the control animal, is indicative of the test animal not being suitable for selective breeding; and wherein the test animal is a monogastric livestock.

In particular, methylation profiling may be used to determine at an early stage of growth of a test animal if the test animal comprises a phenotype of interest or will develop the phenotype of interest at a later stage. Accordingly, rather than raising all animals to adulthood to look for the phenotypes of interest, the method according to this aspect of the present invention may be used to determine at an early stage if the test animal will develop the phenotype of interest. This may save time and resources that are usually required to raise the animals to adulthood. The method according to this aspect of the present invention provides a way to track heritability of epigenetic patterns over time.

The term ‘selective breeding’ as used herein, also known as ‘animal selection’, refers to an animal breeding process carried out by human beings to selectively develop particular phenotypic traits (characteristics) by choosing which typically animal males and females will sexually reproduce and have offspring together. With reference to domesticated animals, this is known as breeds. In animal breeding, techniques such as inbreeding, linebreeding, and outcrossing are utilized. These phenotypic traits also known as ‘phenotype(s) of interest’ as used herein refers to distinct variants of a phenotypic characteristic of an organism that is desired to be present in the selectively bred animal. In particular, the phenotypes of interest in the selectively bred animal are valuable traits that are desired by the breeder to be present in the selectively bred animal. Some examples of phenotype of interest in a cow may be high milk production, quality meat, etc., for chickens, phenotypes of interest may include high production of eggs, meat, and new, young birds for further reproduction, other phenotypes may include feed efficiency (amount of feed required to produce a defined amount of meat/eggs), meat quality characteristics (texture, taste, intramuscular fat), disease resistance, lack of physiological issues (bone, joint or breast meat issues) etc.

According to this aspect of the present invention, the term reference methylation profile may be a panel of methylation profiles where each methylation profile in the panel is related to at least one specific phenotype of interest. For example, in one panel of methylation profiles for chicken, there may be one reference methylation profile for chickens that produce many eggs, there may be one reference methylation profile for chicken with quality meat, there may be one reference methylation profile for chicken being able to reproduce and produce many offspring. Accordingly, the panel of reference methylation profiles for chicken may include one reference methylation profile for each of these phenotypes of interest.

The one or more pre-selected methylation sites in (a) are methylation sites associated with tissue specific gene expression, preferably wherein the pre-selected methylation sites are associated with gene expression of one distinct tissue.

The tissue may be selected from
(i) metabolic tissue such as gut tissue, said gut tissue preferably being ileum or jejunum,
(ii) muscular tissue,
(iii) skin tissue, and
(iv) organ tissue, said organ tissue preferably being hepatic and/or pancreatic tissue.

According to yet another aspect of the present invention, there is provided a DNA methylation-based array for carrying out the method according to any aspect of the present invention.

### BRIEF DESCRIPTION OF FIGURES

FIG. 1 is PCA using differentially methylated positions determined using sequencing in Example 3 where clustering based on Haltungsform is shown.

FIG. 2 are PCA plot results to differentiate between three rearing conditions using the Beadchip data of Example 3.

FIG. 3 is a graph showing that plotting of the first two principal components of the differential methylation analysis CpGs reveal meaningful clustering of the samples for halal and non-halal samples.

FIG. 4 are PCA plots to differentiate between the Halal and Non-halal groups using the Beadchip data.

FIG. 5 is a graph showing that plotting of the first two principal components of the differential methylation analysis CpGs reveal meaningful clustering of the samples from different geographical origins.

FIG. 6 are PCA plots to differentiate between the different locations using the Beadchip data

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

### Geographical Origin Traceability of Salmon Meat

Filet muscle tissues are excised from two-year old Atlantic salmon slaughtered at market weight. Six replicate samples each are obtained from salmon that were reared in either Canada, Chile or Norway.

### DNA Extraction

DNA is extracted using the PureLink Genomic DNA Isolation Minikit kit (Invitrogen), including RNAase treatment following the manufacturer's instructions. DNA quantity is measured by PicoGreen assay and DNA quality is assessed via NanoDrop (Thermo Scientific) to ensure the A260/280 ratio is ≤1.8. A small amount of sample is then also analysed on an agarose gel to ensure each sample contains high molecular weight DNA.

### Bisulfite Conversion and BeadChip Analysis

The genomic DNA samples are then subjected to bisulfite conversion using the EZ DNA Methylation-Gold™ Kit (Zymo Research). The methylation levels are then quantified using our customized methylation BeadChip kits (Illumina) which can analyze over 50,000 methylation sites quantitatively across the genome at single-nucleotide resolution.

### Data Processing:

The customized chip array data processing is performed in R version 4.1.2 using sesame version 1.14.2. DNA methylation level for each site was calculated as methylation β-value. Beta values are defined as methylated signal/(methylated signal+unmethylated signal). It can be computed using getBetas function. The SeSAMe pipeline (Zhou et al. 2018) was used to generate normalized β-values and for quality control. Low intensity-based detection calling and making (based on p-value) was done with pOOBAH. Background subtraction based on normal-exponential deconvolution using out-of-band probes noob (Triche et al. 2013) and optionally with extra bleed-through subtraction were also implemented.

The Differential methylation analysis on the sample groups was also performed using Sesame Results show that the methylation profiles of Salmon meat samples from Canada, Chile or Norway have location dependent methylation profiles, ie. samples coming from the same country are similar to each other, and distinguishable from samples originating from a differing country.

### Example 2

### Confirmation of Halal Certification of Broiler Chicken Meat

Chicken breast muscle meat samples are collected from 6 broilers slaughtered conventionally and 6 broilers slaughtered via Halal certified methods. Halal slaughter of chicken being defined as a chicken that is slaughtered by a single cut across the throat severing both carotid arteries, both jugular veins, both vagus nerves, trachea and esophagus and having been bled to death.

DNA extraction, bisulphite conversion, BeadChip analysis, quality control, data processing and differential methylation analysis are as outlined in Example 1.

Results show that the breast meat from broilers slaughtered via conventional methods have distinct CpG methylation profiles from broilers that are slaughtered with Halal certified methods.

### Example 3

### Assessment of Pork Meat to Confirm Antibiotic Treatment Status in Swine

Back loin meat samples are collected from the carcasses of 6 market weight pigs that were treated orally with Tylosin and 6 similar age and weight pigs that were reared antibiotic-free.

DNA extraction, bisulphite conversion, BeadChip analysis, quality control, data processing and differential methylation analysis are as outlined in Example 1.

Results show that pigs orally treated with Tylosin have distinct methylation profiles in back loin meat DNA when compared to pigs reared in antibiotic-free production.

### Example 4

### Rearing Conditions

### Wet-Lab Methodology

Broiler chicken breast meat was obtained from three different German supermarkets to obtain replicate samples of as many of the German certification standards, known as Haltungsform, as possible. There were four distinct Haltungsform categories for broiler chicken meat in Germany which include Haltungsform 1 (Stallhaltung), 2 (Stallhaltung Plus), 3 (Außenklima) and 4 (Premium). From Haltungsform 1-4 the rearing conditions were improving in alignment with assessed animal welfare needs. For example, space requirements increase from maximum 39 kg of chicken/m² in Stallhaltung to 21 kg/m2 in the Premium category. Additional requirements defining each Haltungsform category include the genetic lines of the broiler chickens, the length of rearing (at last 81 days in Haltungsform 3 and 4), the amount of enrichments and outdoor access available as well as the types of ingredients fed to the animals.

From this sample collection 2 replicate samples of Haltungsform 2 chicken breast meat from each of 3 grocery stores for a total of 6 replicates were obtained, 3 replicate samples of Haltungsform 2 chicken breast meat from 1 grocery store was obtained and 3 replicate samples of Haltungsform 4 chicken breast meat samples from 1 grocery store were obtained. Unfortunately, it was not possible to obtain clearly labelled Haltungsform 1 samples, so this category was excluded. In total there were 12 samples covering 3 of the 4 available Haltungsform categorisations (Table 1).

Table 1. Sample Identification of the 12 Chicken Breast Meat Samples Obtained from 3 of the 4 Distinct Haltungsform Rearing Conditions.

| DNA | sequencing | | | | |
|---|---|---|---|---|---|
| sample id | sample name | Type | Supermarket | Rearing condition | replicate |
| CHKN-0015 | SR_15 | Frozen | Aldi | Haltungsform 2 | Sample 1 |
| CHKN-0016 | SR_16 | Frozen | Aldi | Haltungsform 2 | Sample 2 |
| CHKN-0018 | SR_18 | Frozen | Lidl | Haltungsform 2 | Sample 1 |
| CHKN-0019 | SR_19 | Frozen | Lidl | Haltungsform 2 | Sample 2 |
| CHKN-0021 | SR_21 | Frozen | Rewe | Haltungsform 2 | Sample 1 |
| CHKN-0022 | SR_22 | Frozen | Rewe | Haltungsform 2 | Sample 2 |
| CHKN-0024 | SR_24 | Frozen | Aldi | Haltungsform 3 | Sample 1 |
| CHKN-0025 | SR_25 | Frozen | Aldi | Haltungsform 3 | Sample 2 |
| CHKN-0026 | SR_26 | Frozen | Aldi | Haltungsform 3 | Sample 3 |
| CHKN-0027 | SR_27 | Frozen | Lidl | Haltungsform 4 | Sample 1 |
| CHKN-0028 | SR_28 | Frozen | Lidl | Haltungsform 4 | Sample 2 |
| CHKN-0029 | SR_29 | Frozen | Lidl | Haltungsform 4 | Sample 3 |

### DNA Extraction

DNA is extracted using the PureLink Genomic DNA Isolation Minikit kit (Invitrogen), including RNAase treatment following the manufacturer's instructions. DNA quantity is measured by PicoGreen assay and DNA quality is assessed via NanoDrop (Thermo Scientific) to ensure the A260/280 ratio is ≤1.8. A small amount of sample is then also analysed using automated electrophoresis on TapeStation (Agilent) to ensure each sample contains high molecular weight DNA.

### Sequencing Analysis

Genomic DNA was purified from the breast tissue samples using the DNeasy Blood & Tissue Kit (Qiagen) and is quantified using the PicroGreen or NanoDrop™ 2000.

The genomic DNA (500 ng) from breast tissue samples were used to prepare libraries for Whole Genome Bisulfite Sequencing (WGBS). The sequencing of the libraries was performed by a third party on a NovaSeq platform which generated 125 GB data per sample with 20× coverage.

### Bisulfite Conversion and BeadChip Analysis

A subset of samples were run using the beadchip. There was 2 samples per rearing condition run in triplicates were analyzed.

Table 2. Sample Identification of the 6 Chicken Breast Meat Samples Obtained from 3 of the 4 Distinct Haltungsform Rearing Conditions.

| DNA sample ID | Type | Supermarket | Rearing condition |
|---|---|---|---|
| CHKN-0009 | fresh | Rewe | 2 |
| CHKN-0022 | frozen | Rewe | 2 |
| CHKN-0011 | fresh | Aldi | 3 |
| CHKN-0025 | frozen | Aldi | 3 |
| CHKN-0013 | fresh | Lidl | 4 |
| CHKN-0029 | frozen | Lidl | 4 |

The genomic DNA samples are then subjected to bisulfite conversion using the EZ DNA Methylation-Gold™ Kit (Zymo Research). The methylation levels are then quantified using our customized methylation BeadChip kits (Illumina) which can analyze over 50,000 methylation sites quantitatively across the genome at single-nucleotide resolution. After bisulfite conversion, samples were processed through a three-day workflow including sample amplification, fragmentation, precipitation, hybridization to BeadChip and X-stain according to Infinium HD Methylation Assay (Illumina, Document #15019519 v07), before being imaged on the iScan (Illumina) where intensity files for the computation of beta values are generated.

### Data Processing:

### Processing of Sequencing Data:

Sequenced reads were trimmed and mapped with BSMAP¹ version 2.5 using the assembly version 5.0 of the chicken (Gallus gallus) genome as reference sequence. After deduplication using picard², the methylation ratios were determined using a Python script (methratio.py) distributed with the BSMAP package. For all further analysis, only CpGs covered by at least ten reads were considered, which resulted in 6458063 CpG sites (not provided).

### Differential Methylation Analysis

Differential methylation analysis was performed using MethylKit³ (version 1.12.0) between the different Haltungsform group. MethylKit uses logistic regression to calculate p-values and sliding linear model method⁴ to adjust the p-values to q-values.

CpG sites with an FDR below 0.05 and a methylation change larger than 25% between the groups were considered as significantly differentially methylated Positions (DMPs), resulting in 201246 CpG sites (not provided). methylKit generates a ‘prcomp’ object, which can be used to extract and plot the principal components. Principle Component Analysis (PCA) is a dimensionality-reduction method which can transform large data sets to a few principal components. The first few principal components generally retain most of the variation present in the dataset and are useful for emphasizing the grouping structure in the data

### Processing of Beadchip Data:

The customized chip array data processing is performed in R version 4.1.2 using sesame version 1.14.2. DNA methylation level for each site was calculated as methylation β-value. Beta values are defined as methylated signal/(methylated signal+unmethylated signal). It can be computed using getBetas function. The SeSAMe pipeline (Zhou et al. 2018) was used to generate normalized β-values and for quality control. Low intensity-based detection calling and making (based on p-value) was done with pOOBAH. Background subtraction based on normal-exponential deconvolution using out-of-band probes noob (Triche et al. 2013) and optionally with extra bleed-through subtraction were also implemented. After obtaining the beta value table, probes related to the ‘rearing conditions’ subset were selected, the probes with NA were removed. A PCA plot was plotted for 1517 sites (Table 3a-3g).

TABLE 3
1517 CpG sites on methylation array for determining
rearing conditions for chickens

| | |
|---|---|
| Chrom. | Position |

a

| | |
|---|---|
| chr1 | [symbol] |
| chr1 | [symbol] |
| chr1 | [symbol] |
| chr1 | 61205005 |
| chr1 | [symbol] |
| chr1 | 101481121 |
| chr1 | 168006362 |
| chr1 | 194708326 |
| chr1 | 196005138 |
| chr10 | 1544122 |
| chr11 | [symbol] |
| chr12 | 2951978 |
| chr12 | 18618192 |
| chr13 | [symbol] |
| chr13 | [symbol] |
| chr13 | 178230669 |
| chr14 | [symbol] |
| chr14 | 8154906 |
| chr14 | 13112551 |
| chr15 | 2624238 |
| chr15 | 4983550 |
| chr15 | [symbol] |
| chr17 | 7232737 |
| chr17 | 10435758 |
| chr17 | 10435761 |
| chr18 | 3263783 |
| chr18 | 11027727 |
| chr18 | 11027761 |
| chr19 | 176255 |
| chr19 | 654262 |
| chr19 | [symbol] |
| chr2 | 7859423 |
| chr2 | 23894102 |
| chr2 | [symbol] |
| chr20 | [symbol] |
| chr20 | 10187176 |
| chr20 | 12008400 |
| chr21 | [symbol] |
| chr22 | 457240 |
| chr24 | [symbol] |
| chr25 | [symbol] |
| chr25 | 2734242 |
| chr26 | 1177709 |
| chr26 | 3804165 |
| chr26 | 4883633 |
| chr26 | 5109965 |
| chr28 | 1053019 |
| chr28 | 2555544 |
| chr28 | [symbol] |
| chr3 | 9897964 |
| chr4 | 1826654 |
| chr4 | 2334272 |
| chr4 | 2334294 |
| chr4 | 2334304 |
| chr4 | 2334324 |
| chr4 | 2334380 |
| chr4 | [symbol] |
| chr4 | 39858341 |
| chr4 | 39858423 |
| chr4 | 49833895 |
| chr4 | 79167744 |
| chr5 | 38164423 |
| chr [symbol] | [symbol] |
| chr [symbol] | 19220210 |
| chr [symbol] | 22128574 |
| chr [symbol] | 23106680 |
| chr6 | 23106713 |
| chr7 | 11592904 |
| chr7 | 22544570 |
| chr7 | 36305177 |
| chr7 | 36500183 |
| chr9 | 906245 |
| chr3 | [symbol] |
| chr3 | 36262260 |
| chr3 | 38840553 |
| chr3 | 39178542 |
| chr3 | 40128772 |
| chr3 | 40177015 |
| chr3 | 40692407 |
| chr3 | 40977446 |
| chr3 | [symbol] |
| chr3 | 46066397 |
| chr3 | 48217820 |
| chr3 | 49038833 |
| chr3 | [symbol] |
| chr3 | [symbol] |
| chr3 | [symbol] |
| chr3 | [symbol] |
| chr3 | 56167577 |
| chr3 | [symbol] |
| chr3 | [symbol] |
| chr3 | 57417884 |
| chr3 | [symbol] |
| chr3 | [symbol] |
| chr3 | [symbol] |
| chr3 | 61985549 |
| chr3 | 63130042 |
| chr3 | 63924149 |
| chr3 | 63951433 |
| chr3 | 66133194 |
| chr3 | 67077070 |
| chr3 | 67408583 |
| chr3 | 76030581 |
| chr3 | 76754908 |
| chr3 | 76756047 |
| chr3 | 77142015 |
| chr3 | 77142458 |
| chr3 | 77741058 |
| chr3 | 81340123 |
| chr3 | 82814982 |
| chr3 | [symbol] |
| chr3 | 88775788 |
| chr3 | 89033259 |
| chr3 | 91201007 |
| chr3 | 92951340 |
| chr3 | 96906216 |
| chr3 | 96927964 |
| chr3 | 96928272 |
| chr3 | 97209108 |
| chr3 | 104543262 |
| chr3 | 104905440 |
| chr3 | 105215220 |
| chr3 | 105552987 |
| chr3 | 105636105 |
| chr3 | 105678589 |
| chr3 | 105831487 |
| chr3 | 105977671 |
| chr3 | 106549087 |
| chr3 | 107574089 |
| chr3 | 107849784 |
| chr3 | [symbol] |
| chr3 | 107890733 |
| chr3 | 107911870 |
| chr3 | 108027533 |
| chr3 | [symbol] |
| chr3 | 110159777 |
| chr3 | [symbol] |
| chr33 | 138203 |
| chr33 | 313751 |
| chr33 | 349007 |
| chr33 | 431970 |
| chr33 | 599154 |
| chr33 | 623613 |
| chr33 | 1200939 |
| chr33 | 1245565 |
| chr33 | 1245753 |
| chr33 | 1293081 |
| chr33 | 1592117 |
| chr4 | 910291 |
| chr4 | 1027304 |
| chr4 | 1090281 |
| chr4 | 1233067 |
| chr4 | 1233100 |
| chr4 | 1328712 |
| chr4 | 1421100 |
| chr4 | 1582141 |
| chr4 | [symbol] |
| chr4 | 1601171 |
| chr4 | 1777775 |
| chr4 | 1873993 |
| chr4 | [symbol] |
| chr4 | 1910913 |
| chr4 | 1940154 |
| chr4 | [symbol] |
| chr4 | 2033386 |
| chr4 | 2330437 |
| chr4 | 2330490 |
| chr4 | 2805149 |
| chr4 | 2845160 |
| chr4 | 2855769 |
| chr4 | [symbol] |
| chr4 | [symbol] |
| chr4 | 5154735 |
| chr4 | [symbol] |
| chr4 | 5224952 |
| chr4 | 5232618 |
| chr4 | 5322148 |
| chr4 | [symbol] |
| chr4 | 6438606 |
| chr4 | [symbol] |
| chr4 | [symbol] |
| chr4 | [symbol] |
| chr4 | 9445139 |
| chr4 | 10802703 |
| chr4 | 10966534 |
| chr4 | 10975399 |
| chr4 | 11079713 |
| chr4 | 11091300 |
| chr4 | 11753892 |
| chr4 | 12275738 |
| chr4 | [symbol] |
| chr4 | 12651713 |
| chr4 | [symbol] |
| chr4 | 13478057 |
| chr4 | [symbol] |
| chr4 | 16557157 |
| chr4 | [symbol] |
| chr4 | 16679723 |
| chr4 | 16679754 |
| chr4 | 17722354 |
| chr4 | 17722371 |
| chr4 | 17722410 |
| chr4 | [symbol] |
| chr4 | 18615203 |
| chr4 | 20472363 |
| chr4 | 21041627 |
| chr4 | [symbol] |
| chr4 | [symbol] |
| chr4 | 25777869 |
| chr4 | 29593380 |
| chr4 | 30157188 |
| chr4 | 30434418 |
| chr4 | 30938783 |
| chr4 | [symbol] |
| chr4 | 32571125 |
| chr4 | 32571551 |
| chr4 | [symbol] |
| chr4 | 34420826 |
| chr4 | 34511916 |
| chr4 | 34650792 |
| chr4 | 35249026 |
| chr4 | [symbol] |
| chr4 | 39019297 |
| chr4 | 39640317 |
| chr4 | [symbol] |
| chr4 | 40113483 |
| chr4 | 44658087 |
| chr4 | 45120622 |
| chr4 | 45990171 |
| chr4 | [symbol] |
| chr4 | [symbol] |
| chr4 | 46614360 |
| chr4 | 46744075 |
| chr4 | 46764430 |
| chr4 | [symbol] |
| chr4 | [symbol] |
| chr4 | 49919655 |
| chr4 | [symbol] |
| chr4 | 50351631 |
| chr4 | [symbol] |
| chr4 | 50732937 |
| chr4 | 50738889 |
| chr21 | 2790943 |
| chr21 | 3416693 |
| chr21 | [symbol] |
| chr21 | 4113826 |
| chr21 | 4420716 |
| chr21 | [symbol] |
| chr21 | 4817680 |
| chr21 | 5073216 |

b

| | |
|---|---|
| chr21 | 5134897 |
| chr21 | 5294046 |
| chr21 | 5571658 |
| chr21 | 5576531 |
| chr21 | 6402327 |
| chr22 | 1258890 |
| chr22 | 1441664 |
| chr22 | 2629678 |
| chr22 | 2775225 |
| chr22 | 2875689 |
| chr23 | 205744 |
| chr23 | 1316781 |
| chr23 | 1822864 |
| chr23 | 1870014 |
| chr23 | 2882323 |
| chr23 | [symbol] |
| chr23 | 3219088 |
| chr23 | 3221885 |
| chr23 | 3750032 |
| chr23 | 3794844 |
| chr23 | 4129489 |
| chr23 | 4197051 |
| chr23 | 4327062 |
| chr23 | 4631147 |
| chr23 | [symbol] |
| chr23 | 5521682 |
| chr24 | 316431 |
| chr24 | 352610 |
| chr24 | 380657 |
| chr24 | 521668 |
| chr24 | 3786054 |
| chr24 | 3786074 |
| chr24 | 3791849 |
| chr24 | 4264088 |
| chr24 | 4264381 |
| chr24 | 4452632 |
| chr24 | 4458580 |
| chr24 | 4459949 |
| chr24 | 5001055 |
| chr24 | 5442800 |
| chr24 | 5510032 |
| chr25 | 997002 |
| chr25 | 1174275 |
| chr25 | 1393055 |
| chr25 | 1417052 |
| chr25 | 1447729 |
| chr25 | 1473115 |
| chr25 | 1490009 |
| chr25 | 1733039 |
| chr25 | 1857307 |
| chr25 | 1857358 |
| chr25 | 1873983 |
| chr25 | 2139757 |
| chr25 | 2250098 |
| chr25 | 2499105 |
| chr25 | 2779405 |
| chr25 | 2779506 |
| chr26 | 163655 |
| chr26 | 773746 |
| chr26 | 944256 |
| chr26 | 1186394 |
| chr26 | 1205356 |
| chr26 | 1519097 |
| chr26 | [symbol] |
| chr26 | [symbol] |
| chr26 | [symbol] |
| chr26 | 2617489 |
| chr26 | 2633836 |
| chr26 | 2634425 |
| chr26 | [symbol] |
| chr26 | 3193833 |
| chr26 | 3284013 |
| chr26 | [symbol] |
| chr26 | 3571925 |
| chr26 | 3769919 |
| chr26 | [symbol] |
| chr26 | 3887675 |
| chr26 | 3980660 |
| chr26 | 4006299 |
| chr26 | 4093842 |
| chr26 | 4207730 |
| chr26 | [symbol] |
| chr26 | [symbol] |
| chr26 | 4711384 |
| chr26 | 4711580 |
| chr26 | 4950781 |
| chr26 | 4951644 |
| chr26 | 4984501 |
| chr27 | 9452 |
| chr27 | 9458 |
| chr27 | 450679 |
| chr27 | 450701 |
| chr27 | 450705 |
| chr27 | 450853 |
| chr27 | 535145 |
| chr27 | 549556 |
| chr27 | 568178 |
| chr27 | 1470247 |
| chr27 | 1516484 |
| chr27 | 1690867 |
| chr27 | 1690957 |
| chr27 | 1770333 |
| chr27 | 1830121 |
| chr27 | 2503260 |
| chr27 | 3230431 |
| chr27 | 3579630 |
| chr27 | 3637805 |
| chr27 | 3637822 |
| chr27 | 3878658 |
| chr27 | 4195880 |
| chr27 | [symbol] |
| chr27 | 4696476 |
| chr27 | 4696726 |
| chr27 | 5013394 |
| chr28 | 152879 |
| chr28 | 602855 |
| chr28 | 661354 |
| chr28 | 780718 |
| chr28 | 876393 |
| chr28 | 902555 |
| chr28 | 963297 |
| chr28 | 989287 |
| chr28 | 1477212 |
| chr28 | 1477402 |
| chr28 | 1510646 |
| chr28 | 1756099 |
| chr28 | 2551572 |
| chr28 | 2761422 |
| chr28 | 3244666 |
| chr28 | 3346532 |
| chr28 | 3347239 |
| chr28 | [symbol] |
| chr28 | 3450035 |
| chr28 | 3881563 |
| chr28 | 3994803 |
| chr28 | 4112479 |
| chr28 | 4493216 |
| chr28 | 4493486 |
| chr28 | 4601551 |
| chr28 | 4601787 |
| chr3 | 762248 |
| chr3 | 3041542 |
| chr3 | 3106361 |
| chr3 | 3222435 |
| chr3 | 3227462 |
| chr3 | 3709394 |
| chr3 | 3709412 |
| chr3 | 3709414 |
| chr3 | 4693557 |
| chr3 | 4747083 |
| chr3 | 7307719 |
| chr3 | 7314763 |
| chr3 | 7620832 |
| chr3 | 7621056 |
| chr3 | 7795011 |
| chr3 | 8076823 |
| chr3 | 8106957 |
| chr3 | 8540353 |
| chr3 | 9111239 |
| chr3 | [symbol] |
| chr3 | 10043478 |
| chr3 | 10402191 |
| chr3 | 10646459 |
| chr3 | 12468131 |
| chr3 | 12863377 |
| chr3 | 13114795 |
| chr3 | 13764816 |
| chr3 | 13776514 |
| chr3 | 13777106 |
| chr3 | 13879323 |
| chr3 | 14213694 |
| chr3 | 14692554 |
| chr3 | 15378622 |
| chr3 | 15397056 |
| chr3 | 15429434 |
| chr3 | 15654896 |
| chr3 | 16473660 |
| chr3 | 16597276 |
| chr3 | 16990982 |
| chr3 | 17151889 |
| chr3 | [symbol] |
| chr3 | [symbol] |
| chr3 | 17641637 |
| chr3 | 17931353 |
| chr3 | 19547328 |
| chr3 | 21125603 |
| chr3 | 21810958 |
| chr3 | 21843637 |
| chr3 | 22223021 |
| chr3 | 22702842 |
| chr3 | 23979475 |
| chr3 | 24950104 |
| chr3 | 25287789 |
| chr3 | 27383343 |
| chr3 | 30310480 |
| chr3 | 30336758 |
| chr3 | 31855962 |
| chr3 | 31856154 |
| chr2 | 49207688 |
| chr2 | 49207740 |
| chr2 | 56377762 |
| chr2 | 56838448 |
| chr2 | 58027095 |
| chr2 | 60570579 |
| chr2 | 60719489 |
| chr2 | 60719498 |
| chr2 | 60762017 |
| chr2 | 61679021 |
| chr2 | 61925404 |
| chr2 | [symbol] |
| chr2 | 62121274 |
| chr2 | 63095683 |
| chr2 | 63182554 |
| chr2 | 65246730 |
| chr2 | 65776014 |
| chr2 | [symbol] |
| chr2 | [symbol] |
| chr2 | 67882807 |
| chr2 | 78593271 |
| chr2 | 78805450 |
| chr2 | 78945165 |
| chr2 | 80105433 |
| chr2 | 84574600 |
| chr2 | 89047434 |
| chr2 | 89347564 |
| chr2 | 96956810 |
| chr2 | 98768413 |
| chr2 | 99076619 |
| chr2 | 99082338 |
| chr2 | 99085675 |
| chr2 | [symbol] |
| chr2 | 100407546 |
| chr2 | 100407597 |
| chr2 | 100480189 |
| chr2 | [symbol] |
| chr2 | 102774453 |
| chr2 | 102822416 |
| chr2 | 102994576 |
| chr2 | 109960124 |
| chr2 | 110356873 |
| chr2 | 111613464 |
| chr2 | 111960015 |
| chr2 | 111960400 |
| chr2 | 114813030 |
| chr2 | 115378040 |
| chr2 | 115378117 |
| chr2 | 115378408 |
| chr2 | 115378430 |
| chr2 | 115673685 |
| chr2 | 115692267 |

c

| | |
|---|---|
| chr2 | 115921454 |
| chr2 | 121315243 |
| chr2 | 122036210 |
| chr2 | 123333539 |
| chr2 | 124732349 |
| chr2 | 125171977 |
| chr2 | 125171980 |
| chr2 | [symbol] |
| chr2 | 127761601 |
| chr2 | 127977964 |
| chr2 | 129161729 |
| chr2 | 129510254 |
| chr2 | 129823473 |
| chr2 | 129925091 |
| chr2 | [symbol] |
| chr2 | 136001970 |
| chr2 | 138197264 |
| chr2 | 138262185 |
| chr2 | 138458022 |
| chr2 | 139418387 |
| chr2 | 142740341 |
| chr2 | 145744047 |
| chr2 | 145760186 |
| chr2 | 149040559 |
| chr2 | 149057704 |
| chr2 | 149267598 |
| chr2 | 149298315 |
| chr2 | 149428970 |
| chr2 | 149459170 |
| chr20 | 579180 |
| chr20 | 100718 |
| chr20 | 1000763 |
| chr20 | 1107235 |
| chr20 | 1125502 |
| chr20 | 1215112 |
| chr20 | 1276429 |
| chr20 | 3445305 |
| chr20 | 3793389 |
| chr20 | [symbol] |
| chr20 | 5272517 |
| chr20 | 5307675 |
| chr20 | 5366847 |
| chr20 | 5394896 |
| chr20 | 5470992 |
| chr20 | 5556570 |
| chr20 | 5711779 |
| chr20 | 5722442 |
| chr20 | 5756009 |
| chr20 | 5756119 |
| chr20 | 6882326 |
| chr20 | 7247140 |
| chr20 | 8145390 |
| chr20 | 8177346 |
| chr20 | 8632238 |
| chr20 | 9192038 |
| chr20 | 9253454 |
| chr20 | 9455282 |
| chr20 | 9455284 |
| chr20 | 9533918 |
| chr20 | 9618439 |
| chr20 | 9830540 |
| chr20 | 10275863 |
| chr20 | 10275971 |
| chr20 | 10299625 |
| chr20 | 10313730 |
| chr20 | 10549709 |
| chr20 | 10712150 |
| chr20 | 10712265 |
| chr20 | 10714422 |
| chr20 | 10805733 |
| chr20 | 11771486 |
| chr20 | 11771861 |
| chr20 | 13293728 |
| chr20 | 13676694 |
| chr21 | 757314 |
| chr21 | 927350 |
| chr21 | 1483434 |
| chr21 | 2196326 |
| chr21 | 2220700 |
| chr21 | 2540383 |
| chr21 | 2589747 |
| chr21 | 2594608 |
| chr21 | 2632160 |
| chr18 | 9968570 |
| chr18 | 10158720 |
| chr18 | 10194005 |
| chr18 | 10432543 |
| chr18 | 11027653 |
| chr18 | 11027657 |
| chr18 | 11027665 |
| chr19 | [symbol] |
| chr19 | 193722 |
| chr19 | 304423 |
| chr19 | 443591 |
| chr19 | 641464 |
| chr19 | 641791 |
| chr19 | 641964 |
| chr19 | [symbol] |
| chr19 | 730334 |
| chr19 | 730801 |
| chr19 | 785877 |
| chr19 | 3402216 |
| chr19 | 3402669 |
| chr19 | [symbol] |
| chr19 | 3779599 |
| chr19 | 4324099 |
| chr19 | 4492763 |
| chr19 | 4492948 |
| chr19 | 4919139 |
| chr19 | 4919379 |
| chr19 | 5038641 |
| chr19 | 5039039 |
| chr19 | 5129851 |
| chr19 | 6020757 |
| chr19 | [symbol] |
| chr19 | 6244690 |
| chr19 | 7209175 |
| chr19 | 7242371 |
| chr19 | 8696771 |
| chr2 | 302611 |
| chr2 | 337050 |
| chr2 | 483870 |
| chr2 | 535538 |
| chr2 | 847486 |
| chr2 | 963741 |
| chr2 | 2290908 |
| chr2 | 3969845 |
| chr2 | 6220902 |
| chr2 | 6549601 |
| chr2 | 6549620 |
| chr2 | 6814842 |
| chr2 | 7859358 |
| chr2 | 13012909 |
| chr2 | 13974558 |
| chr2 | 14241834 |
| chr2 | 14643959 |
| chr2 | 16222160 |
| chr2 | 16730652 |
| chr2 | 17735001 |
| chr2 | 17751064 |
| chr2 | 18083028 |
| chr2 | 19883286 |
| chr2 | 20363551 |
| chr2 | 22164304 |
| chr2 | 23152655 |
| chr2 | 23153430 |
| chr2 | 23943414 |
| chr2 | 32724069 |
| chr2 | 34373790 |
| chr2 | 37342351 |
| chr2 | 38076907 |
| chr2 | [symbol] |
| chr2 | [symbol] |
| chr2 | 39048661 |
| chr2 | [symbol] |
| chr2 | 40777422 |
| chr2 | 40936047 |
| chr2 | 41252040 |
| chr2 | 41841626 |
| chr2 | 42279756 |
| chr2 | 42335751 |
| chr2 | 46929396 |
| chr2 | 47605446 |
| chr2 | [symbol] |
| chr9 | 906999 |
| chr9 | 1045853 |
| chr9 | 1125703 |
| chr9 | 1277906 |
| chr9 | [symbol] |
| chr9 | [symbol] |
| chr9 | 2084974 |
| chr9 | [symbol] |
| chr9 | 3371714 |
| chr9 | 3805239 |
| chr9 | 4768076 |
| chr9 | 4868302 |
| chr9 | 5006812 |
| chr9 | 5274273 |
| chr9 | 5274300 |
| chr9 | 5404054 |
| chr9 | 6645664 |
| chr9 | 6856009 |
| chr9 | 7627284 |
| chr9 | 7627307 |
| chr9 | 7627312 |
| chr9 | 7627315 |
| chr9 | 7627369 |
| chr9 | 7968195 |
| chr9 | 7968206 |
| chr9 | 8174477 |
| chr9 | 8472863 |
| chr9 | 8748045 |
| chr9 | 10619172 |
| chr9 | 10619341 |
| chr9 | 10619447 |
| chr9 | 10673083 |
| chr9 | 11487183 |
| chr9 | [symbol] |
| chr9 | 13319040 |
| chr9 | 14072942 |
| chr9 | 14306768 |
| chr9 | 14524879 |
| chr9 | 14971851 |
| chr9 | 15015159 |
| chr9 | 15205844 |
| chr9 | 15268157 |
| chr9 | 15338077 |
| chr9 | 15438693 |
| chr9 | 15615697 |
| chr9 | 15647871 |
| chr9 | 15664766 |
| chr9 | 15732480 |
| chr9 | 15739172 |
| chr9 | 15874216 |
| chr9 | 16044699 |
| chr9 | 16580797 |
| chr9 | 17679739 |
| chr9 | 19518525 |
| chr9 | 21878826 |
| chr13 | 13224396 |
| chr13 | 13224498 |
| chr13 | 13224693 |
| chr13 | 13471280 |
| chr13 | 14499771 |
| chr13 | 16749513 |
| chr13 | 16930934 |
| chr13 | 17164837 |
| chr13 | 17230981 |
| chr13 | 17352331 |
| chr13 | 17375184 |
| chr13 | 17650342 |
| chr13 | 17705605 |
| chr13 | 41834 |
| chr14 | 706228 |
| chr14 | 860171 |
| chr14 | 1666470 |
| chr14 | [symbol] |
| chr14 | 2603496 |
| chr14 | 3084073 |
| chr14 | 4450196 |
| chr14 | 4468140 |
| chr14 | 4552136 |
| chr14 | 4552282 |
| chr14 | 5029916 |
| chr14 | 5059196 |
| chr14 | 5137701 |
| chr14 | 5262069 |
| chr14 | 5262419 |
| chr14 | 5647143 |
| chr14 | 5662253 |

d

| | |
|---|---|
| chr14 | 6075809 |
| chr14 | 6131371 |
| chr14 | 6339673 |
| chr14 | 6596177 |
| chr14 | 6596571 |
| chr14 | 6696623 |
| chr14 | [symbol] |
| chr14 | 8217252 |
| chr14 | 8217810 |
| chr14 | [symbol] |
| chr14 | 8690350 |
| chr14 | 10043625 |
| chr14 | 12156025 |
| chr14 | 12169797 |
| chr14 | 12325261 |
| chr14 | 12371322 |
| chr14 | 12466354 |
| chr14 | 12511829 |
| chr14 | 12811447 |
| chr14 | 13343507 |
| chr14 | 13473402 |
| chr14 | 14060876 |
| chr14 | 14256248 |
| chr14 | 14289568 |
| chr14 | 14984644 |
| chr15 | 713522 |
| chr15 | 966307 |
| chr15 | 1478772 |
| chr15 | 1642934 |
| chr15 | 2239610 |
| chr15 | 4517727 |
| chr15 | 4684620 |
| chr15 | 5027220 |
| chr15 | 5039827 |
| chr15 | 5106785 |
| chr15 | 5470661 |
| chr15 | 5489704 |
| chr15 | 6316549 |
| chr15 | 6974901 |
| chr15 | 7040187 |
| chr15 | 7044175 |
| chr15 | 7044278 |
| chr15 | 7347540 |
| chr15 | 7571659 |
| chr15 | 7896557 |
| chr15 | 7978480 |
| chr15 | 8024717 |
| chr15 | 8621534 |
| chr15 | 8949976 |
| chr15 | 8973339 |
| chr15 | 9117481 |
| chr15 | 9143777 |
| chr15 | 9231258 |
| chr15 | 9299857 |
| chr15 | 9320092 |
| chr15 | 9414904 |
| chr15 | 9763212 |
| chr15 | [symbol] |
| chr15 | 9945103 |
| chr15 | 10043653 |
| chr15 | 10867537 |
| chr15 | 10916474 |
| chr15 | 10963998 |
| chr15 | 12233766 |
| chr16 | 83772 |
| chr16 | 85532 |
| chr16 | 96406 |
| chr17 | 1310045 |
| chr17 | 1414407 |
| chr17 | 2360182 |
| chr17 | 4501515 |
| chr17 | 5420538 |
| chr17 | 6027806 |
| chr17 | 6140808 |
| chr17 | 6190184 |
| chr17 | 6338381 |
| chr17 | 6423489 |
| chr17 | 6543625 |
| chr17 | 6609861 |
| chr17 | 6717101 |
| chr17 | 6934951 |
| chr17 | 7232953 |
| chr17 | 7299085 |
| chr17 | 7422482 |
| chr17 | 7464258 |
| chr17 | 7628308 |
| chr17 | 7904953 |
| chr17 | 8205987 |
| chr17 | 8376816 |
| chr17 | 8376829 |
| chr17 | 8754431 |
| chr17 | 8793017 |
| chr17 | 8919833 |
| chr17 | 8990882 |
| chr17 | 9171058 |
| chr17 | 9309814 |
| chr17 | 9928751 |
| chr17 | 9983756 |
| chr17 | 10284613 |
| chr17 | 10468830 |
| chr18 | 705748 |
| chr18 | 1501510 |
| chr18 | 1856301 |
| chr18 | 2211597 |
| chr18 | 2212594 |
| chr18 | 2212698 |
| chr18 | 2219152 |
| chr18 | 3188421 |
| chr18 | 4109304 |
| chr18 | 4333561 |
| chr18 | 4681896 |
| chr18 | 6083995 |
| chr18 | 6382250 |
| chr18 | 6504687 |
| chr18 | 6559744 |
| chr18 | 7373781 |
| chr18 | 7373876 |
| chr18 | 7374222 |
| chr18 | 7987501 |
| chr18 | 9097594 |
| chr18 | 9315699 |
| chr18 | 9385760 |
| chr18 | 9583270 |
| chr18 | 9734925 |
| chr18 | 9843044 |
| chr18 | 9889887 |
| chr1 | 167764091 |
| chr1 | 167764721 |
| chr1 | 169365529 |
| chr1 | 169378645 |
| chr1 | 169399944 |
| chr1 | 169486963 |
| chr1 | 169497713 |
| chr1 | 170482101 |
| chr1 | 171260545 |
| chr1 | 171967903 |
| chr1 | 171971350 |
| chr1 | 174547104 |
| chr1 | 176202314 |
| chr1 | 176202804 |
| chr1 | 177056332 |
| chr1 | 177418702 |
| chr1 | [symbol] |
| chr1 | 180247788 |
| chr1 | [symbol] |
| chr1 | 182580926 |
| chr1 | 184976532 |
| chr1 | 185524691 |
| chr1 | 187683828 |
| chr1 | 188768818 |
| chr1 | 189165861 |
| chr1 | 190445290 |
| chr1 | [symbol] |
| chr1 | 192858910 |
| chr1 | 192939826 |
| chr1 | 192953625 |
| chr1 | 193821130 |
| chr1 | 194535301 |
| chr1 | 195334826 |
| chr1 | 196197590 |
| chr10 | 778251 |
| chr10 | 1257330 |
| chr10 | 1463709 |
| chr10 | 1504262 |
| chr10 | 1520583 |
| chr10 | 1532993 |
| chr10 | 2141375 |
| chr10 | 2333800 |
| chr10 | 2373574 |
| chr10 | 2480593 |
| chr10 | 2500145 |
| chr10 | 2764991 |
| chr10 | 2987858 |
| chr10 | 4066675 |
| chr10 | 6806633 |
| chr10 | 7930834 |
| chr10 | 7930856 |
| chr10 | 8001614 |
| chr10 | 8631532 |
| chr10 | 8828241 |
| chr10 | 9002142 |
| chr10 | 9002495 |
| chr10 | 10075337 |
| chr10 | 11406434 |
| chr10 | 12564139 |
| chr10 | 12859290 |
| chr10 | 13032542 |
| chr10 | 13200332 |
| chr10 | 13386585 |
| chr10 | 14341408 |
| chr10 | 15878758 |
| chr10 | 17409198 |
| chr10 | 17848338 |
| chr10 | 18174931 |
| chr10 | 18493084 |
| chr10 | 18529280 |
| chr10 | 18543626 |
| chr10 | 19239921 |
| chr10 | 19246087 |
| chr10 | 19393141 |

e

| | |
|---|---|
| chr11 | 500386 |
| chr11 | 648948 |
| chr11 | 943468 |
| chr11 | 1199004 |
| chr11 | 1223129 |
| chr11 | 1257168 |
| chr11 | [symbol] |
| chr11 | 1920839 |
| chr11 | 6303597 |
| chr11 | 6303882 |
| chr11 | 8241481 |
| chr11 | [symbol] |
| chr11 | 10055912 |
| chr11 | 10194539 |
| chr11 | [symbol] |
| chr11 | 14165094 |
| chr11 | 15791007 |
| chr11 | 15791023 |
| chr11 | 17141970 |
| chr11 | 18010566 |
| chr11 | 18172296 |
| chr11 | 18788822 |
| chr11 | 19011572 |
| chr11 | 19085781 |
| chr11 | [symbol] |
| chr11 | 19191933 |
| chr11 | 19262052 |
| chr12 | 594371 |
| chr12 | 710870 |
| chr12 | 1006248 |
| chr12 | 1416724 |
| chr12 | 1781167 |
| chr12 | 2315019 |
| chr12 | 2402107 |
| chr12 | 2459692 |
| chr12 | 2832340 |
| chr12 | 2832375 |
| chr12 | [symbol] |
| chr12 | 2952000 |
| chr12 | 3231935 |
| chr12 | 3499160 |
| chr12 | 3499346 |
| chr12 | 3499411 |
| chr12 | [symbol] |
| chr12 | [symbol] |
| chr12 | 6363727 |
| chr12 | [symbol] |
| chr12 | 6636292 |
| chr12 | 6759129 |
| chr12 | 7055347 |
| chr12 | 9157101 |
| chr12 | [symbol] |
| chr12 | 9326187 |
| chr12 | 9675444 |
| chr12 | 10134215 |
| chr12 | 10471466 |
| chr12 | 10691612 |
| chr12 | 10720347 |
| chr12 | 11522531 |
| chr12 | 11550264 |
| chr12 | 11550339 |
| chr12 | [symbol] |
| chr12 | 11571999 |
| chr12 | 11597987 |
| chr12 | 11620774 |
| chr12 | [symbol] |
| chr12 | 11688762 |
| chr12 | 11689429 |
| chr12 | 13353292 |
| chr12 | 13658758 |
| chr12 | 15316510 |
| chr12 | 15614180 |
| chr12 | 19373964 |
| chr12 | 19520055 |
| chr12 | 19554314 |
| chr13 | 1075586 |
| chr13 | 1714487 |
| chr13 | [symbol] |
| chr13 | 4689571 |
| chr13 | 4706956 |
| chr13 | 10027100 |
| chr13 | 11063260 |
| chr13 | 11469004 |
| chr13 | 12880006 |
| chr13 | 13224232 |
| chr7 | 11045520 |
| chr7 | 12037727 |
| chr7 | 12181772 |
| chr7 | 14200735 |
| chr7 | 15878913 |
| chr7 | 15906091 |
| chr7 | 16281087 |
| chr7 | 16435357 |
| chr7 | 16868670 |
| chr7 | [symbol] |
| chr7 | 17492185 |
| chr7 | 17584266 |
| chr7 | 18582014 |
| chr7 | 19230886 |
| chr7 | 19534058 |
| chr7 | 20168248 |
| chr7 | 22211846 |
| chr7 | 22211898 |
| chr7 | 22449124 |
| chr7 | 22722508 |
| chr7 | 22947604 |
| chr7 | 23871704 |
| chr7 | 24179280 |
| chr7 | 25387013 |
| chr7 | 27097005 |
| chr8 | 2027164 |
| chr8 | 2308395 |
| chr8 | 2308524 |
| chr8 | 2651114 |
| chr8 | 2651725 |
| chr8 | [symbol] |
| chr8 | 3872497 |
| chr8 | 4305613 |
| chr8 | 4928505 |
| chr8 | 5643261 |
| chr8 | 7547903 |
| chr8 | [symbol] |
| chr8 | 8038099 |
| chr8 | 13597810 |
| chr8 | 14403655 |
| chr8 | 14713760 |
| chr8 | 16296124 |
| chr8 | [symbol] |
| chr8 | 16539699 |
| chr8 | 16589375 |
| chr8 | 16729492 |
| chr8 | 18749436 |
| chr8 | 19872627 |
| chr8 | [symbol] |
| chr8 | [symbol] |
| chr8 | 20612357 |
| chr8 | 20653624 |
| chr8 | 20807126 |
| chr8 | 20807804 |
| chr8 | 21330680 |
| chr8 | 21330737 |
| chr8 | 21581249 |
| chr8 | 24611572 |
| chr8 | 24731314 |
| chr8 | [symbol] |
| chr8 | [symbol] |
| chr8 | [symbol] |
| chr8 | 25350826 |
| chr8 | 26434110 |
| chr8 | 26707714 |
| chr8 | 27412358 |
| chr8 | 28360528 |
| chr8 | 28360600 |
| chr8 | 29104689 |
| chr8 | 29384546 |
| chr8 | 29520307 |
| chr9 | 708289 |
| chr7 | 27444943 |
| chr7 | [symbol] |
| chr7 | 28224043 |
| chr7 | 28273161 |
| chr7 | [symbol] |
| chr7 | [symbol] |
| chr7 | 28368958 |
| chr7 | 28564743 |
| chr7 | 30784070 |
| chr7 | 31558200 |
| chr7 | 31558280 |
| chr7 | 31658345 |
| chr7 | 33793326 |
| chr7 | [symbol] |
| chr7 | 35302141 |
| chr7 | 35330918 |
| chr7 | 36417944 |
| chr7 | 36448560 |
| chr8 | 1513867 |
| chr8 | 1659896 |
| chr8 | 1659986 |
| chr1 | 144446585 |
| chr1 | 144525743 |
| chr1 | 144596000 |
| chr1 | 144628599 |
| chr1 | 144806925 |
| chr1 | 144806941 |
| chr1 | 144936599 |
| chr1 | 145127674 |
| chr1 | [symbol] |
| chr1 | 153175508 |
| chr1 | 156111598 |
| chr1 | 165873085 |
| chr5 | 35942840 |
| chr5 | 35951948 |
| chr5 | 38034094 |
| chr5 | 38247217 |
| chr5 | 39028943 |
| chr5 | 39239801 |
| chr5 | 43854507 |
| chr5 | 43916169 |
| chr5 | 44158031 |
| chr5 | 45377462 |
| chr5 | [symbol] |
| chr5 | 46696711 |
| chr5 | 48259193 |
| chr5 | 48261657 |
| chr5 | 48282420 |
| chr5 | [symbol] |
| chr5 | 51614514 |
| chr5 | 52256208 |
| chr5 | 52974612 |
| chr5 | 52999403 |
| chr5 | 53496100 |
| chr5 | 53813947 |
| chr5 | 53874774 |
| chr5 | 54214379 |
| chr5 | [symbol] |
| chr5 | 56598068 |
| chr5 | 56612257 |
| chr5 | 56816701 |
| chr5 | 57121115 |
| chr5 | 57631314 |
| chr5 | 57931939 |
| chr5 | [symbol] |
| chr5 | 58156156 |
| chr5 | 58214675 |
| chr5 | 58214995 |
| chr5 | 58997382 |
| chr6 | 2328508 |
| chr6 | 3002937 |
| chr6 | 3003006 |
| chr6 | [symbol] |
| chr6 | 5064205 |
| chr6 | 5270603 |
| chr6 | 8082174 |
| chr6 | 9169855 |
| chr6 | 9607324 |
| chr6 | [symbol] |
| chr6 | 10819414 |
| chr6 | 10965280 |
| chr6 | 11176399 |
| chr6 | 11215952 |
| chr6 | 11504681 |
| chr6 | 11509043 |
| chr6 | 11517878 |
| chr6 | 11587843 |
| chr6 | 11795446 |
| chr6 | 12006696 |
| chr6 | 14931644 |
| chr6 | 16993318 |
| chr6 | 17825108 |
| chr6 | 21198024 |

f

| | |
|---|---|
| chr6 | 21495793 |
| chr6 | 22239320 |
| chr6 | 22317215 |
| chr6 | 22542975 |
| chr6 | [symbol] |
| chr6 | 23080151 |
| chr6 | 23639995 |
| chr6 | 23640014 |
| chr6 | 23640090 |
| chr6 | 23640236 |
| chr6 | 24194953 |
| chr6 | 25417973 |
| chr6 | 25977368 |
| chr6 | 30055057 |
| chr6 | 30728819 |
| chr6 | 30729306 |
| chr6 | 31409434 |
| chr6 | 31444814 |
| chr6 | 32081463 |
| chr6 | 32083522 |
| chr6 | 32327367 |
| chr6 | 32533531 |
| chr6 | [symbol] |
| chr6 | 32595338 |
| chr6 | 32651984 |
| chr6 | 33644967 |
| chr6 | 34225180 |
| chr6 | 34594737 |
| chr7 | 301344 |
| chr7 | 301591 |
| chr7 | 301604 |
| chr7 | 301817 |
| chr7 | 1121452 |
| chr7 | 2778648 |
| chr7 | 2817987 |
| chr7 | 2818039 |
| chr7 | 2818041 |
| chr7 | 3785133 |
| chr7 | 4542360 |
| chr7 | 5039298 |
| chr7 | 5782548 |
| chr7 | 10229207 |
| chr7 | 10607236 |
| chr4 | 53929550 |
| chr4 | 57532120 |
| chr4 | [symbol] |
| chr4 | 60116333 |
| chr4 | 60369319 |
| chr4 | 61442447 |
| chr4 | 63175291 |
| chr4 | 63498697 |
| chr4 | 63913908 |
| chr4 | 64974927 |
| chr4 | 65597747 |
| chr4 | 65835628 |
| chr4 | 65835907 |
| chr4 | 66593188 |
| chr4 | 66871450 |
| chr4 | 67254323 |
| chr4 | 68043996 |
| chr1 | 845041 |
| chr1 | 845430 |
| chr1 | 944313 |
| chr1 | 1135242 |
| chr1 | 5762768 |
| chr1 | 6327061 |
| chr1 | 6996280 |
| chr1 | 12721673 |
| chr1 | 13959611 |
| chr1 | 14382900 |
| chr1 | 14979250 |
| chr1 | 15342645 |
| chr1 | 15721124 |
| chr1 | 16473870 |
| chr1 | 17860898 |
| chr1 | 24806572 |
| chr1 | 25090652 |
| chr1 | 25543855 |
| chr1 | 26913555 |
| chr1 | 28733309 |
| chr1 | [symbol] |
| chr1 | [symbol] |
| chr1 | 35484379 |
| chr1 | 35485237 |
| chr1 | 35519037 |
| chr1 | 36508947 |
| chr1 | 38315968 |
| chr1 | 43648094 |
| chr1 | 43996630 |
| chr1 | 45631451 |
| chr1 | 45634725 |
| chr1 | 45775764 |
| chr1 | 45911015 |
| chr1 | 47927053 |
| chr1 | 47949158 |
| chr1 | 48224103 |
| chr1 | 48250341 |
| chr1 | 48281644 |
| chr1 | [symbol] |
| chr1 | 48390193 |
| chr1 | 48868613 |
| chr1 | 49197441 |
| chr1 | 49237049 |
| chr1 | 49260504 |
| chr1 | 49510358 |
| chr1 | 49510470 |
| chr1 | 49689947 |
| chr1 | 49713524 |
| chr1 | 49770584 |
| chr1 | 49788265 |
| chr1 | 49788463 |
| chr1 | 49788647 |
| chr1 | 49906819 |
| chr1 | 49923743 |
| chr1 | 49965259 |
| chr1 | [symbol] |
| chr1 | 50198598 |
| chr1 | 50347759 |
| chr1 | 50348093 |
| chr1 | 50393671 |
| chr1 | 5037038 |
| chr1 | 50837347 |
| chr1 | 51173893 |
| chr1 | 51227977 |
| chr1 | 51266323 |
| chr1 | 51266370 |
| chr1 | 51290294 |
| chr1 | 52202655 |
| chr1 | 53851570 |
| chr1 | 55562267 |
| chr1 | 55723811 |
| chr1 | 55832872 |
| chr1 | 56378388 |
| chr1 | 56606536 |
| chr1 | 56859045 |
| chr1 | 58996570 |
| chr1 | 59481718 |
| chr1 | 59625186 |
| chr1 | 60250579 |
| chr1 | 61159125 |
| chr1 | 61217619 |
| chr1 | 61708337 |
| chr1 | 61854947 |
| chr1 | 62208396 |
| chr1 | 62231636 |
| chr1 | 62659212 |
| chr1 | 64820820 |
| chr1 | 65301984 |
| chr1 | 65578611 |
| chr1 | 65578725 |
| chr1 | 67313718 |
| chr1 | 67373896 |
| chr1 | 67374283 |
| chr1 | 67608276 |
| chr1 | 67824141 |
| chr1 | 68304376 |
| chr1 | 68407787 |
| chr1 | 68964661 |
| chr1 | 69134414 |
| chr1 | 70415016 |
| chr1 | 71724800 |
| chr1 | 77585690 |
| chr1 | 77816964 |
| chr1 | 78022840 |
| chr1 | 79368010 |
| chr1 | 79410556 |
| chr1 | 79917796 |
| chr1 | 80157976 |
| chr1 | 82492311 |
| chr1 | 83053516 |
| chr1 | 83670149 |
| chr1 | 84409963 |
| chr1 | 84850177 |
| chr1 | 85256473 |
| chr1 | 87489932 |
| chr1 | [symbol] |
| chr1 | 88379452 |
| chr1 | 88379501 |
| chr1 | 88405537 |
| chr1 | 88976273 |
| chr1 | 910212499 |
| chr1 | 91544655 |
| chr1 | [symbol] |
| chr1 | 92290840 |
| chr1 | 92291341 |
| chr1 | 92330191 |
| chr1 | 92494362 |
| chr1 | 92494374 |
| chr1 | 98590523 |
| chr1 | 99018003 |
| chr1 | 104038164 |
| chr1 | 104068101 |
| chr1 | 104410439 |
| chr1 | 105319904 |
| chr1 | 105838735 |
| chr1 | [symbol] |
| chr1 | 109095178 |
| chr1 | 109696785 |
| chr1 | 109727485 |
| chr1 | 109790935 |
| chr1 | 10237440 |
| chr1 | 110237964 |
| chr1 | 110650502 |
| chr1 | 112198174 |
| chr1 | 112267538 |
| chr1 | 112769186 |
| chr1 | 113326083 |
| chr1 | 115769298 |
| chr1 | 118222959 |
| chr1 | 118360164 |
| chr1 | 120318726 |
| chr1 | 120318954 |
| chr1 | 122985528 |
| chr1 | 124631488 |
| chr1 | 124980421 |
| chr1 | 125132312 |
| chr1 | 126123458 |
| chr1 | 128712082 |
| chr1 | 128887957 |
| chr1 | 130278246 |
| chr1 | 130278359 |
| chr1 | 130394035 |
| chr1 | 130466685 |
| chr1 | 132541877 |
| chr1 | 133288255 |
| chr1 | 134117703 |
| chr4 | 68572135 |
| chr4 | 68787977 |
| chr4 | [symbol] |
| chr4 | 77471918 |
| chr4 | 80579347 |
| chr4 | 81350777 |
| chr4 | 82546871 |
| chr4 | 82901266 |
| chr4 | 83040845 |
| chr4 | 85215159 |
| chr4 | 86247587 |
| chr4 | 86247854 |
| chr4 | 86248148 |
| chr4 | 86269569 |
| chr4 | 86401069 |
| chr4 | 86456226 |
| chr4 | 86535966 |
| chr4 | 86645773 |
| chr4 | 86748832 |
| chr4 | [symbol] |
| chr4 | 88894964 |
| chr4 | 89258617 |
| chr4 | 89775627 |
| chr4 | 91259068 |

g

| | |
|---|---|
| chr5 | 37485 |
| chr5 | 37494 |
| chr5 | 266410 |
| chr5 | 285012 |
| chr5 | 291581 |
| chr5 | 292685 |
| chr5 | 405296 |
| chr5 | 480120 |
| chr5 | 530650 |
| chr5 | 535230 |
| chr5 | 829463 |
| chr5 | 1006724 |
| chr5 | 1006936 |
| chr5 | [symbol] |
| chr5 | 1352126 |
| chr5 | 1513368 |
| chr5 | 1640571 |
| chr5 | 7832284 |
| chr5 | 8797294 |
| chr5 | 10085372 |
| chr5 | 10407965 |
| chr5 | 11621859 |
| chr5 | 12576421 |
| chr5 | 13242466 |
| chr5 | 14140634 |
| chr5 | 14715578 |
| chr5 | 14754447 |
| chr5 | 14840213 |
| chr5 | 15821492 |
| chr5 | 15888309 |
| chr5 | 16038883 |
| chr5 | 16620665 |
| chr5 | 16871510 |
| chr5 | 18393880 |
| chr5 | 19083382 |
| chr5 | 19003715 |
| chr5 | 19129484 |
| chr5 | 19157790 |
| chr5 | 19507409 |
| chr5 | 19522196 |
| chr5 | 19522392 |
| chr5 | 19522409 |
| chr5 | 21647478 |
| chr5 | 23105105 |
| chr5 | 23453492 |
| chr5 | 23921159 |
| chr5 | 23939029 |
| chr5 | 24199704 |
| chr5 | 24437840 |
| chr5 | 24791744 |
| chr5 | 25025576 |
| chr5 | 25026274 |
| chr5 | [symbol] |
| chr5 | 25026320 |
| chr5 | 25520233 |
| chr5 | 27300901 |
| chr5 | 28318927 |
| chr5 | 28804112 |
| chr5 | 29494916 |
| chr5 | 30090062 |
| chr5 | [symbol] |
| chr5 | 30627044 |
| chr5 | 30728316 |
| chr5 | [symbol] |
| chr5 | 34188428 |
| chrLGE64 | 152490 |
| chrLGE64 | 248401 |

[symbol]

indicates data missing or illegible when filed

### Results

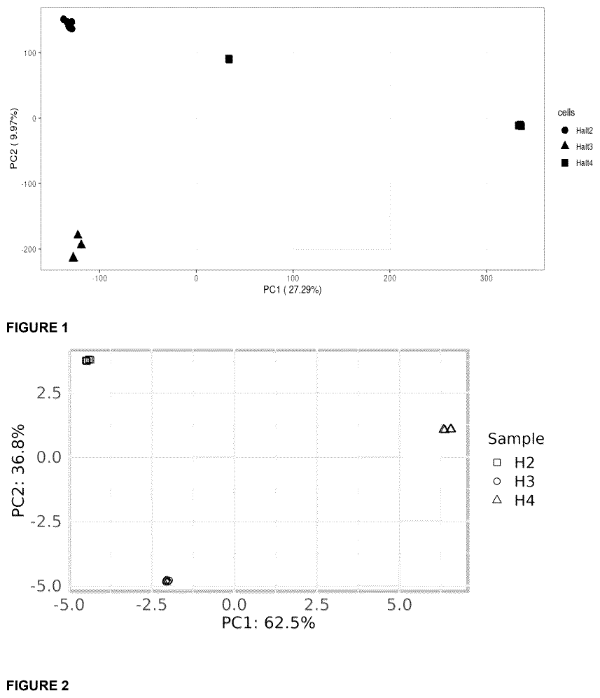

When a second PCA was run using the differentially methylated positions this clustering based on Haltungsform, otherwise known as rearing condition, became even clearer (FIG. 1).

PCA plots was run to differentiate between three rearing conditions using the Beadchip data (FIG. 2).

In summary, plotting of the first two principal components of the CpGs reveal meaningful clustering of the samples processed by sequencing and beadchip data. CpG sites were able to effectively cluster of the different groups in a meaningful way. We can effectively replicate the results of the Haltungsform application with the beadchip.

### Example 5

### Halal

25 Wet-Lab methodology Halal and non halal chicken breast meat were purchased from five different vendors. The vendors for halal meat include Pasar, ZAC Butchery, Hego premium, RedMart and Keesong, whereas non halal meat was purchased from Pasar, Tegel, Farm fresh, Ryan and AW's in Singapore.

| | |
|---|---|
| Halal | Non-Halal |
| Brand | Brand |
| Pasar | Pasar |
| ZAC Butchery | Tegel |
| Hego premium | Farm fresh |
| Red Mart | Ryan |
| Keesong | AW's |

DNA Extraction, Sequencing Analysis, Bisulfite Conversion and BeadChip Analysis and data processing are carried out according to example 4.

After obtaining the beta value table for the samples, probes related to the ‘Halal’ subset were selected, the probes with NA were removed. A PCA plot was plotted for 506 sites (Table 4)

TABLE 4
506 CpG sites on the DNA methylation array for determining halal or not halal

| Chrom. | Position | Chrom. | Position | Chrom. | Position | Chrom. | Position | Chrom. | Position |
|---|---|---|---|---|---|---|---|---|---|
| chr1 | 12877821 | chr3 | 76507235 | chr26 | 1651982 | chr2 | 127781801 | chr2 | 42358641 |
| chr1 | 14903055 | chr3 | 76739615 | chr26 | 2590932 | chr2 | 129925091 | chr2 | 42913387 |
| chr1 | 15441151 | chr3 | 76739886 | chr26 | 3248923 | chr2 | 129933052 | chr2 | 46089114 |
| chr1 | 39877658 | chr3 | 76755038 | chr26 | 3571402 | chr2 | 130013087 | chr2 | 46089139 |
| chr1 | 50488695 | chr3 | 77142645 | chr26 | 4858960 | chr20 | 533436 | chr2 | 49110506 |
| chr1 | 56825511 | chr3 | 96481436 | chr26 | 5003532 | chr20 | 1000718 | chr9 | 4626749 |
| chr1 | 76766536 | chr3 | 102971530 | chr27 | 1541894 | chr20 | 4533305 | chr9 | 4676234 |
| chr10 | 10680859 | chr3 | 104543849 | chr27 | 3464008 | chr20 | 5394896 | chr9 | 4850885 |
| chr10 | 12500816 | chr3 | 104905440 | chr27 | 3637822 | chr20 | 5519899 | chr9 | 5089285 |
| chr10 | 15213509 | chr3 | 105215220 | chr27 | 4719697 | chr20 | 5579795 | chr9 | 5216234 |
| chr11 | 6629490 | chr3 | 105678589 | chr27 | 4720035 | chr20 | 8145679 | chr9 | 6855496 |
| chr12 | 15606143 | chr3 | 105678613 | chr28 | 153360 | chr20 | 9983514 | chr9 | 7624289 |
| chr17 | 7463984 | chr3 | 105678619 | chr28 | 153426 | chr20 | 10287258 | chr9 | 7724217 |
| chr17 | 10895495 | chr3 | 107890733 | chr28 | 1477408 | chr20 | 10511507 | chr9 | 8479123 |
| chr18 | 8977183 | chr3 | 108708555 | chr28 | 3642946 | chr21 | 927717 | chr9 | 9528198 |
| chr19 | 176255 | chr33 | 431970 | chr28 | 3800027 | chr21 | 1483434 | chr9 | 9539267 |
| chr19 | 1401235 | chr4 | 1985540 | chr3 | 2421530 | chr21 | 2332272 | chr9 | 10594523 |
| chr2 | 78548181 | chr4 | 2123054 | chr3 | 7315133 | chr21 | 2332328 | chr9 | 10619341 |
| chr2 | 121381306 | chr4 | 5202992 | chr3 | 8952808 | chr18 | 10185868 | chr9 | 10672938 |
| chr21 | 2688710 | chr4 | 6740626 | chr3 | 12583058 | chr19 | 50552 | chr9 | 14072787 |
| chr23 | 4152128 | chr4 | 8695640 | chr3 | 12755149 | chr19 | 185280 | chr9 | 15414814 |
| chr24 | 375697 | chr4 | 10802755 | chr3 | 12755221 | chr19 | 199882 | chr9 | 15812625 |
| chr24 | 4885733 | chr4 | 11719574 | chr3 | 12789564 | chr19 | 580436 | chr9 | 22079838 |
| chr28 | 2443299 | chr4 | 21041627 | chr3 | 19170252 | chr19 | 2411046 | chr9 | 22225808 |
| chr3 | 4193791 | chr4 | 32571055 | chr3 | 21854776 | chr19 | 4088736 | chrZ | 7668187 |
| chr5 | 24369896 | chr4 | 34288785 | chr3 | 22702842 | chr19 | 4492763 | chrZ | 11047446 |
| chr5 | 39173222 | chr4 | 39012274 | chr3 | 26344201 | chr19 | 5363084 | chrZ | 11287708 |
| chr6 | 1549721 | chr4 | 39536841 | chr3 | 26860812 | chr19 | 5384384 | chrZ | 11287812 |
| chr7 | 11248389 | chr4 | 39537311 | chr3 | 27258633 | chr19 | 5767147 | chrZ | 16048197 |
| chr7 | 22569247 | chr4 | 39771776 | chr3 | 30399012 | chr19 | 5832481 | chrZ | 18103001 |
| chr8 | 16295920 | chr4 | 40113483 | chr3 | 31394938 | chr19 | 5832942 | chrZ | 17123376 |
| chr8 | 28088175 | chr4 | 43808241 | chr2 | 57948024 | chr19 | 6022617 | chrZ | 24913814 |
| chr8 | 29363057 | chr4 | 45296016 | chr2 | 62413006 | chr19 | 7203329 | chrZ | 27177202 |
| chr9 | 7925956 | chr4 | 45296889 | chr2 | 66497871 | chr19 | 8481362 | chrZ | 27177213 |
| chr3 | 32261768 | chr4 | 46058642 | chr2 | 78548594 | chr19 | 8867093 | chrZ | 27270228 |
| chr3 | 32362994 | chr4 | 49890681 | chr2 | 85904873 | chr2 | 1390782 | chrZ | 27444073 |
| chr3 | 33946497 | chr21 | 4836900 | chr2 | 88162809 | chr2 | 3856589 | chrZ | 33678917 |
| chr3 | 38638958 | chr21 | 5073396 | chr2 | 91651196 | chr2 | 4575039 | chrZ | 34328034 |
| chr3 | 38237516 | chr23 | 1870014 | chr2 | 93915362 | chr2 | 4980847 | chrZ | 37529420 |
| chr3 | 38336190 | chr23 | 2381593 | chr2 | 102562607 | chr2 | 5096922 | chrZ | 63907320 |
| chr3 | 39148221 | chr23 | 3739407 | chr2 | 111960409 | chr2 | 13471116 | chrZ | 65981131 |
| chr3 | 40601306 | chr24 | 804341 | chr2 | 114090502 | chr2 | 15482005 | chrZ | 72829308 |
| chr3 | 45066858 | chr24 | 3786074 | chr2 | 115673685 | chr2 | 20693997 | chrZ | 61360739 |
| chr3 | 46372522 | chr24 | 4459949 | chr2 | 115921454 | chr2 | 20877142 | chr13 | 16065472 |
| chr3 | 48423158 | chr25 | 976468 | chr2 | 118638998 | chr2 | 21010987 | chr13 | 17037566 |
| chr3 | 49813062 | chr25 | 1107409 | chr2 | 121422611 | chr2 | 21041245 | chr13 | 18308448 |
| chr3 | 51285929 | chr25 | 1876704 | chr2 | 121482517 | chr2 | 23153225 | chr14 | 957744 |
| chr3 | 58674863 | chr25 | 1946574 | chr2 | 123206194 | chr2 | 26782587 | chr14 | 2268610 |
| chr3 | 65294828 | chr26 | 1519097 | chr2 | 124732645 | chr2 | 34511190 | chr14 | 5000387 |
| chr3 | 67720493 | chr26 | 1544666 | chr2 | 125171980 | chr2 | 41841626 | chr14 | 5059398 |
| chr14 | 5261997 | chr1 | 188916642 | chr7 | 15587386 | chr6 | 10819506 | | |
| chr14 | 6596177 | chr1 | 193821130 | chr7 | 16281087 | chr6 | 11221033 | | |
| chr14 | 6696623 | chr1 | 194236534 | chr7 | 17289720 | chr6 | 11517878 | | |
| chr14 | 6926191 | chr1 | 194401733 | chr7 | 18690074 | chr6 | 11915935 | | |
| chr14 | 7327840 | chr1 | 195334826 | chr7 | 22245891 | chr6 | 12121122 | | |
| chr14 | 8217810 | chr10 | 967899 | chr7 | 22755583 | chr6 | 12121146 | | |
| chr14 | 10043625 | chr10 | 2315851 | chr7 | 24170000 | chr6 | 17322989 | | |
| chr14 | 12811447 | chr10 | 2333800 | chr8 | 2118531 | chr6 | 22239587 | | |
| chr15 | 5027241 | chr10 | 3477178 | chr8 | 6089739 | chr6 | 24169226 | | |
| chr15 | 5823857 | chr10 | 4390201 | chr8 | 6818970 | chr6 | 27262610 | | |
| chr15 | 5989722 | chr10 | 6806714 | chr8 | 7548136 | chr6 | 28147880 | | |
| chr15 | 6891797 | chr10 | 10824700 | chr8 | 11915537 | chr6 | 32236852 | | |
| chr15 | 7003924 | chr10 | 10895845 | chr8 | 13384214 | chrê | 32533539 | | |
| chr15 | 7347328 | chr10 | 11406434 | chr8 | 14713737 | chr7 | 301591 | | |
| chr15 | 7571659 | chr10 | 12400953 | chr8 | 16052167 | chr7 | 301604 | | |
| chr15 | 8003087 | chr10 | 12734127 | chr8 | 16296168 | chr7 | 2239686 | | |
| chr15 | 9763222 | chr10 | 18678519 | chr8 | 16370376 | chr7 | 5077349 | | |
| chr16 | 168962 | chr11 | 841570 | chr8 | 16374372 | chr7 | 6603676 | | |
| chr17 | 1980788 | chr11 | 1459001 | chr8 | 16589375 | chr7 | 9697869 | | |
| chr17 | 2360133 | chr11 | 6629712 | chr8 | 16729492 | chr7 | 9938317 | | |
| chr17 | 2935626 | chr11 | 10194539 | chr8 | 25610078 | chr4 | 53533077 | | |
| chr17 | 4062722 | chr11 | 10256374 | chr8 | 28653132 | chr4 | 57532309 | | |
| chr17 | 5741333 | chr11 | 10452379 | chr7 | 28179710 | chr4 | 57572893 | | |
| chr17 | 5799733 | chr11 | 10578956 | chr7 | 28205381 | chr4 | 58554384 | | |
| chr17 | 6189674 | chr11 | 10950629 | chr7 | 29790478 | chr4 | 60368609 | | |
| chr17 | 6765666 | chr11 | 11215844 | chr7 | 35189129 | chr4 | 60369319 | | |
| chr17 | 7673395 | chr11 | 18172296 | chr7 | 35847537 | chr4 | 66670233 | | |
| chr17 | 7904953 | chr11 | 18808803 | chr8 | 1401347 | chr1 | 3700593 | | |
| chr17 | 8203614 | chr11 | 18808917 | chr1 | 137293780 | chr1 | 7558524 | | |
| chr17 | 8205987 | chr12 | 710959 | chr1 | 144628599 | chr1 | 14382900 | | |
| chr17 | 8376829 | chr12 | 1581083 | chr1 | 144796538 | chr1 | 16473870 | | |
| chr17 | 8377056 | chr12 | 1581381 | chr1 | 144796614 | chr1 | 24855132 | | |
| chr17 | 9978114 | chr12 | 2259754 | chr1 | 153736291 | chr1 | 26767903 | | |
| chr17 | 10895665 | chr12 | 2372012 | chr1 | 153739829 | chr1 | 34377737 | | |
| chr18 | 1708416 | chr12 | 4380982 | chr1 | 165873023 | chr1 | 35104436 | | |
| chr18 | 2274676 | chr12 | 6363727 | chr1 | 165873085 | chr1 | 35484379 | | |
| chr18 | 3699658 | chr12 | 7012484 | chr5 | 38166099 | chr1 | 36508947 | | |
| chr18 | 3700112 | chr12 | 7055474 | chr5 | 39113705 | chr1 | 39858634 | | |
| chr18 | 3700175 | chr12 | 9875444 | chr5 | 43891403 | chr1 | 39877753 | | |
| chr18 | 3777721 | chr12 | 10134215 | chr5 | 48066943 | chr1 | 44860990 | | |
| chr18 | 7373876 | chr12 | 10148968 | chr5 | 48084582 | chr1 | 44871524 | | |
| chr18 | 9435327 | chr12 | 11951638 | chr5 | 51772714 | chr1 | 47926496 | | |
| chr1 | 168855019 | chr13 | 3283054 | chr5 | 52943641 | chr1 | 48250341 | | |
| chr1 | 169234551 | chr13 | 6729058 | chr5 | 52943689 | chr1 | 49441925 | | |
| chr1 | 169374378 | chr13 | 11458665 | chr5 | 53477916 | chr1 | 49770596 | | |
| chr1 | 175460817 | chr13 | 12290255 | chr5 | 53496100 | chr1 | 50703527 | | |
| chr1 | 175460839 | chr13 | 13028604 | chr5 | S3918500 | chr1 | 50837347 | | |
| chr1 | 175461006 | chr13 | 13224232 | chr5 | 58997382 | chr1 | 51088644 | | |
| chr1 | 175806942 | chr7 | 11358921 | chr8 | 9407093 | chr1 | 53092288 | | |
| chr1 | 178990900 | chr7 | 15096160 | chr8 | 10819414 | chr1 | 53953094 | | |
| chr1 | 54593907 | chrS_NT_460419v1_random | 6746 | | | | | | |
| chr1 | 56521314 | chrUn_NT_464142v1 | 64936 | | | | | | |
| chr1 | 56606169 | chrUn_NT_464199v1 | 28427 | | | | | | |
| chr1 | 01708721 | chrUn_NT_464303v1 | S736 | | | | | | |
| chr1 | 65578611 | chrUn_NT_465658v1 | 7191 | | | | | | |
| chr1 | 65578655 | chrUn_NT_468071v1 | 11202 | | | | | | |
| chr1 | 65576725 | chrUn_NT_471028v1 | 3312 | | | | | | |
| chr1 | 68455273 | | | | | | | | |
| chr1 | 71113923 | | | | | | | | |
| chr1 | 76766539 | | | | | | | | |
| chr1 | 77229485 | | | | | | | | |
| chr1 | 79199586 | | | | | | | | |
| chr1 | 80519994 | | | | | | | | |
| chr1 | 83226834 | | | | | | | | |
| chr1 | 84409963 | | | | | | | | |
| chr1 | 84532408 | | | | | | | | |
| chr1 | 88411659 | | | | | | | | |
| chr1 | 88759238 | | | | | | | | |
| chr1 | 91422737 | | | | | | | | |
| chr1 | 92271976 | | | | | | | | |
| chr1 | 92330716 | | | | | | | | |
| chr1 | 108957860 | | | | | | | | |
| chr1 | 110237976 | | | | | | | | |
| chr1 | 110880942 | | | | | | | | |
| chr1 | 113283028 | | | | | | | | |
| chr1 | 122962624 | | | | | | | | |
| chr1 | 123415519 | | | | | | | | |
| chr1 | 130278359 | | | | | | | | |
| chr1 | 130973501 | | | | | | | | |
| chr4 | 73695235 | | | | | | | | |
| chr4 | 86456090 | | | | | | | | |
| chr4 | 88992493 | | | | | | | | |
| chr4 | 88992878 | | | | | | | | |
| chr4 | 89259160 | | | | | | | | |
| chr5 | 480120 | | | | | | | | |
| chr5 | 9279301 | | | | | | | | |
| chr5 | 9737337 | | | | | | | | |
| chr5 | 10355756 | | | | | | | | |
| chr5 | 11091055 | | | | | | | | |
| chr5 | 11621859 | | | | | | | | |
| chr5 | 12488676 | | | | | | | | |
| chr5 | 13107975 | | | | | | | | |
| chr5 | 14162871 | | | | | | | | |
| chr5 | 19156349 | | | | | | | | |
| chr5 | 23458500 | | | | | | | | |
| chr5 | 27885744 | | | | | | | | |
| chr5 | 28115369 | | | | | | | | |
| chr5 | 28318496 | | | | | | | | |
| chr5 | 29057117 | | | | | | | | |

### Results

Plotting of the first two principal components of the CpGs before and after differential methylation analysis reveal meaningful clustering of the samples. Differentially methylated Positions (DMPs) were able to effectively cluster of the halal and non-halal groups in a meaningful way.

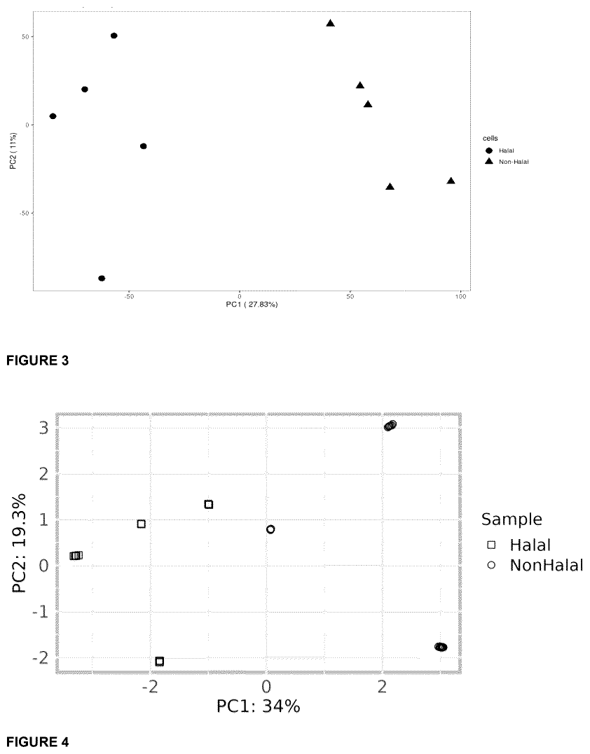

Plotting of the first two principal components of the differential methylation analysis CpGs reveal meaningful clustering of the samples (FIG. 3).

PCA plots was run to differentiate between the Halal and Non-halal groups using the Beadchip data (FIG. 4).

In summary, plotting of the first two principal components of the CpGs reveal meaningful clustering of the samples processed by sequencing and beadchip data. CpG sites were able to effectively cluster of the different groups in a meaningful way. We can effectively replicate the results of the Halal application with the beadchip.

### Example 6

### Origin Traceability—Identification of Differentially Methylated CpG Sites in Chicken

In order to identify differentially methylated CpG sites in the chicken, the function “calculate 5 DiffMeth” from the R package MethylKit was used on the Reduced representation bisulfite sequencing (RRBS) data.

### Material and Methods

Isolated and purified genomic DNA from breast muscular tissue was provided by different service laboratories in the respective country of sample source. Quality was checked using a 2200 TapeStation (Agilent).

RRBS library preparation was carried out as described in the Zymo-Seq RRBS™ Library Kit Instruction Manual Ver. 1.0.0. Quality controls were performed, and sample concentrations were measured on a 2200 TapeStation (Agilent). Multiplexed samples were sequenced on a HiSeq 4000 system (Illumina).

Reads were quality trimmed using trimmomatic version 0.38 and mapped with BSMAP 2.90 to the Gallus gallus genome assembly version 5.0. Methylation ratios were calculated using a python script (methratio.py) distributed with the BSMAP package. All the CpG sites that were associated with sex chromosomes and the CpG sites that overlapped with SNPs for the Gallus gallus genome 25 were filtered out from the further analysis. Prior to this analysis, the data was filtered for SNPs and a coverage cutoff of minimum 10 per CpG site was applied. Differential methylation analysis was performed using the R package MethylKit (Akalin et al. (2012), Genome Biology, 13(10), R87).

MethylKit uses logistic regression to calculate p-values and sliding linear model method to adjust the p-values to q-values. CpG sites with an FDR below 0.05 and a methylation change larger than 25% between the groups were considered as significantly differentially methylated Positions (DMPs), resulting in 8500 CpG sites.

MethylKit generates a ‘prcomp’ object, which can be used to extract and plot the principal components. The use of MethylKit is disclosed in Akalin A, et al. Genome Biol. 2012; 13(10): R87. Principle Component Analysis (PCA) is a dimensionality-reduction method which can transform large data sets to a few principal components. The first few principal components generally retain most of the variation present in the dataset and are useful for emphasizing the grouping structure in the data

Bisulfite Conversion and BeadChip Analysis and Processing of Beadchip data are carried out according to Example 3.

After obtaining the beta value table for the samples, probes related to the ‘Origin Tracebility’ subset were selected, the probes with NA were removed. From the remaining probes, the variance for each probe was calculated. A PCA plot was also created using the top 1000 probes (out of 12466 probes). (Table 5)

TABLE 5
top 1000 probes (out of 12466 probes) used in the chip for determining geographic origin

| Chrom. | Position | Chrom. | Position | Chrom. | Position | Chrom. | Position | Chrom. | Position |
|---|---|---|---|---|---|---|---|---|---|
| chr1 | 417356 | chr1 | 178820994 | chr11 | 4306865 | chr12 | 11580828 | chr13 | 17382178 |
| chr1 | 429451 | chr1 | 185455678 | chr11 | 6978609 | chr12 | 11581077 | chr13 | 17993922 |
| chr1 | 601241 | chr1 | 185510269 | chr11 | 7976003 | chr12 | 11620945 | chr14 | 926048 |
| chr1 | 711820 | chr1 | 186711540 | chr11 | 8423829 | chr12 | 13680719 | chr14 | 1294970 |
| chr1 | 6144280 | chr1 | 186711654 | chr11 | 8601372 | chr12 | 13783688 | chr14 | 2122082 |
| chr1 | 7141107 | chr1 | 192596011 | chr11 | 10355069 | chr12 | 15606143 | chr14 | 2654082 |
| chr1 | 15441151 | chr1 | 194233909 | chr11 | 12804335 | chr12 | 15934547 | chr14 | 3163366 |
| chr1 | 15898266 | chr1 | 194363116 | chr11 | 12804350 | chr12 | 16013686 | chr14 | 3221086 |
| chr1 | 20209666 | chr1 | 194463402 | chr11 | 14504066 | chr12 | 16430242 | chr14 | 3957685 |
| chr1 | 33706411 | chr1 | 194606852 | chr11 | 15519889 | chr12 | 16778219 | chr14 | 4155990 |
| chr1 | 34832598 | chr1 | 194665704 | chr11 | 17898779 | chr12 | 17331988 | chr14 | 4342490 |
| chr1 | 35822015 | chr1 | 194707084 | chr11 | 17910668 | chr12 | 17810143 | chr14 | 4431434 |
| chr1 | 44887243 | chr1 | 194818482 | chr11 | 18611325 | chr12 | 18115135 | chr14 | 4475471 |
| chr1 | 48224417 | chr1 | 196057345 | chr11 | 18786742 | chr12 | 18531367 | chr14 | 4679001 |
| chr1 | 49249845 | chr10 | 1325060 | chr11 | 18796943 | chr12 | 18699814 | chr14 | 4684588 |
| chr1 | 49457178 | chr10 | 1351836 | chr11 | 18802209 | chr12 | 19441726 | chr14 | 4949821 |
| chr1 | 49525264 | chr10 | 1462851 | chr11 | 18804677 | chr13 | 1011839 | chr14 | 5041018 |
| chr1 | 50488695 | chr10 | 1504701 | chr11 | 18894011 | chr13 | 1057121 | chr14 | 5063938 |
| chr1 | 51011667 | chr10 | 1954836 | chr11 | 18993963 | chr13 | 1358962 | chr14 | 5169655 |
| chr1 | 51155986 | chr10 | 2278255 | chr11 | 19061426 | chr13 | 1714849 | chr14 | 5403463 |
| chr1 | 51173894 | chr10 | 2692051 | chr11 | 19110543 | chr13 | 1714851 | chr14 | 5781935 |
| chr1 | 51175428 | chr10 | 2751219 | chr11 | 19715975 | chr13 | 1715958 | chr14 | 5877992 |
| chr1 | 59988862 | chr10 | 2752805 | chr12 | 1005950 | chr13 | 1715957 | chr14 | 6112752 |
| chr1 | 60959799 | chr10 | 12500816 | chr12 | 1448421 | chr13 | 1715964 | chr14 | 6417476 |
| chr1 | 61847133 | chr10 | 16839689 | chr12 | 1477421 | chr13 | 2150049 | chr14 | 7200759 |
| chr1 | 63446136 | chr10 | 18703406 | chr12 | 1495387 | chr13 | 2432965 | chr14 | 12284518 |
| chr1 | 68570871 | chr10 | 19049484 | chr12 | 1763012 | chr13 | 2432973 | chr14 | 12730383 |
| chr1 | 69177355 | chr10 | 19172166 | chr12 | 1859363 | chr13 | 2432978 | chr14 | 12755878 |
| chr1 | 76590233 | chr10 | 19326407 | chr12 | 2356541 | chr13 | 2434775 | chr14 | 12762302 |
| chr1 | 76590243 | chr10 | 19380347 | chr12 | 2407800 | chr13 | 2435762 | chr14 | 13058684 |
| chr1 | 78663677 | chr10 | 19615869 | chr12 | 2458439 | chr13 | 2591920 | chr14 | 13112649 |
| chr1 | 80158333 | chr10 | 19882898 | chr12 | 2462569 | chr13 | 3188075 | chr14 | 13533317 |
| chr1 | 80839115 | chr10 | 19980035 | chr12 | 2462581 | chr13 | 8494961 | chr14 | 13638383 |
| chr1 | 81937300 | chr10 | 20011081 | chr12 | 2562223 | chr13 | 10144056 | chr14 | 13762590 |
| chr1 | 92230188 | chr10 | 20019644 | chr12 | 2562309 | chr13 | 10145965 | chr14 | 14087403 |
| chr1 | 98553688 | chr10 | 20026593 | chr12 | 2844671 | chr13 | 10160015 | chr14 | 14140237 |
| chr1 | 101229928 | chr10 | 20048135 | chr12 | 3170920 | chr13 | 10160027 | chr14 | 14141456 |
| chr1 | 104660033 | chr10 | 20051189 | chr12 | 3210564 | chr13 | 10168366 | chr14 | 14151641 |
| chr1 | 105349994 | chr10 | 20067260 | chr12 | 3233154 | chr13 | 10191695 | chr14 | 14167458 |
| chr1 | 108016932 | chr10 | 20094709 | chr12 | 6389853 | chr13 | 10201356 | chr15 | 709123 |
| chr1 | 112007740 | chr10 | 20112737 | chr12 | 6466349 | chr13 | 10249423 | chr15 | 1766475 |
| chr1 | 112386033 | chr11 | 534157 | chr12 | 6614911 | chr13 | 10290188 | chr15 | 1843516 |
| chr1 | 117835444 | chr11 | 609072 | chr12 | 9103014 | chr13 | 10293259 | chr15 | 3365499 |
| chr1 | 144538327 | chr11 | 650395 | chr12 | 9969774 | chr13 | 10336326 | chr15 | 4674381 |
| chr1 | 163224006 | chr11 | 1039778 | chr12 | 11315945 | chr13 | 10344357 | chr15 | 7141462 |
| chr1 | 170269386 | chr11 | 1046349 | chr12 | 11485337 | chr13 | 10457049 | chr15 | 7182219 |
| chr1 | 175182758 | chr11 | 1104215 | chr12 | 11516587 | chr13 | 13111818 | chr15 | 7372208 |
| chr1 | 175196058 | chr11 | 1384387 | chr12 | 11520338 | chr13 | 15750949 | chr15 | 7517064 |
| chr1 | 176293589 | chr11 | 1459014 | chr12 | 11572654 | chr13 | 15891776 | chr15 | 7990520 |
| chr1 | 176293632 | chr11 | 1768577 | chr12 | 11575933 | chr13 | 17043376 | chr15 | 8298478 |
| chr15 | 8443051 | chr18 | 4469748 | chr19 | 806178 | chr2 | 24485287 | chr20 | 13022291 |
| chr15 | 10916114 | chr18 | 6010125 | chr19 | 1394840 | chr2 | 38272762 | chr20 | 13481945 |
| chr15 | 11243256 | chr18 | 6489780 | chr19 | 1401236 | chr2 | 41462172 | chr21 | 667339 |
| chr15 | 11264561 | chr18 | 8720760 | chr19 | 2524036 | chr2 | 44056938 | chr21 | 1190625 |
| chr15 | 11346422 | chr18 | 8977183 | chr19 | 4073220 | chr2 | 47854647 | chr21 | 1286227 |
| chr15 | 11518021 | chr18 | 9046067 | chr19 | 4248507 | chr2 | 56441954 | chr21 | 2627311 |
| chr16 | 62976 | chr18 | 9049042 | chr19 | 4253772 | chr2 | 57949078 | chr21 | 2676843 |
| chr16 | 67707 | chr18 | 9124024 | chr19 | 4346245 | chr2 | 57949172 | chr21 | 2688710 |
| chr16 | 79149 | chr18 | 9190686 | chr19 | 4880430 | chr2 | 61859824 | chr21 | 2769409 |
| chr16 | 114874 | chr18 | 9223316 | chr19 | 5342776 | chr2 | 61972505 | chr21 | 3937147 |
| chr16 | 215563 | chr18 | 9228239 | chr19 | 5379921 | chr2 | 67226378 | chr21 | 3366958 |
| chr17 | 904530 | chr18 | 9275751 | chr19 | 5450174 | chr2 | 86909829 | chr21 | 4207802 |
| chr17 | 956038 | chr18 | 9001037 | chr19 | 5614688 | chr2 | 89362067 | chr21 | 4306933 |
| chr17 | 1059357 | chr18 | 9311341 | chr19 | 5798940 | chr2 | 97949789 | chr21 | 4332229 |
| chr17 | 1187604 | chr18 | 9332799 | chr19 | 5880900 | chr2 | 102881150 | chr21 | 4891009 |
| chr17 | 1236995 | chr18 | 9363906 | chr19 | 5915123 | chr2 | 104135640 | chr21 | 6029810 |
| chr17 | 1236046 | chr18 | 9369994 | chr19 | 5919056 | chr2 | 107587594 | chr21 | 6087082 |
| chr17 | 1232214 | chr18 | 9579784 | chr19 | 6063293 | chr2 | 107822425 | chr21 | 6538478 |
| chr17 | 1958875 | chr18 | 9581235 | chr19 | 7611676 | chr2 | 110662522 | chr21 | 6743064 |
| chr17 | 2457103 | chr18 | 9605762 | chr19 | 7682271 | chr2 | 111706209 | chr22 | 457240 |
| chr17 | 2458797 | chr18 | 9643102 | chr19 | 7746755 | chr2 | 115378315 | chr22 | 1288350 |
| chr17 | 3814145 | chr18 | 9687520 | chr19 | 8392052 | chr2 | 123313721 | chr22 | 1630200 |
| chr17 | 3825134 | chr18 | 9702417 | chr19 | 8919570 | chr2 | 121381306 | chr22 | 2358528 |
| chr17 | 5132859 | chr18 | 9757765 | chr19 | 8987526 | chr2 | 127348938 | chr22 | 2970967 |
| chr17 | 5324534 | chr18 | 9841715 | chr19 | 9645825 | chr2 | 133838796 | chr22 | 4641216 |
| chr17 | 6077236 | chr18 | 9879969 | chr2 | 207656 | chr2 | 137932547 | chr22 | 4642845 |
| chr17 | 6103243 | chr18 | 9887792 | chr2 | 214585 | chr2 | 138598584 | chr23 | 1822865 |
| chr17 | 6605830 | chr18 | 9895042 | chr2 | 217497 | chr2 | 138972712 | chr23 | 1823644 |
| chr17 | 6608917 | chr18 | 9908250 | chr2 | 217686 | chr2 | 149519088 | chr23 | 1896943 |
| chr17 | 7732455 | chr18 | 9948577 | chr2 | 230369 | chr20 | 569930 | chr23 | 1899919 |
| chr17 | 8050861 | chr18 | 9954378 | chr2 | 230606 | chr20 | 595711 | chr23 | 2026074 |
| chr17 | 8776503 | chr18 | 10145516 | chr2 | 244732 | chr20 | 3314840 | chr23 | 2032864 |
| chr17 | 9779024 | chr18 | 10373359 | chr2 | 252343 | chr20 | 4330862 | chr23 | 2068566 |
| chr17 | 9781180 | chr18 | 10384158 | chr2 | 272028 | chr20 | 5216107 | chr23 | 2183679 |
| chr17 | 9919240 | chr18 | 10427581 | chr2 | 274295 | chr20 | 5734109 | chr23 | 2239934 |
| chr17 | 10007118 | chr18 | 10514006 | chr2 | 324380 | chr20 | 7666555 | chr23 | 3478983 |
| chr17 | 1011527 | chr18 | 10703553 | chr2 | 324383 | chr20 | 9542324 | chr23 | 4069126 |
| chr17 | 10142204 | chr18 | 11027686 | chr2 | 386444 | chr20 | 9688000 | chr23 | 4145394 |
| chr17 | 10176653 | chr18 | 11027714 | chr2 | 433099 | chr20 | 9819948 | chr23 | 4150616 |
| chr17 | 10243408 | chr18 | 11027727 | chr2 | 517076 | chr20 | 9337256 | chr23 | 4150634 |
| chr17 | 10284611 | chr18 | 11027736 | chr2 | 560878 | chr20 | 10090892 | chr23 | 4151462 |
| chr18 | 1824783 | chr18 | 11027742 | chr2 | 596166 | chr20 | 10100654 | chr23 | 4152128 |
| chr18 | 2087048 | chr18 | 13027758 | chr2 | 3887651 | chr20 | 10262951 | chr23 | 4165993 |
| chr18 | 2093726 | chr18 | 11027761 | chr2 | 4084824 | chr20 | 10429308 | chr23 | 4308042 |
| chr18 | 2260268 | chr19 | 171631 | chr2 | 4085817 | chr20 | 10438912 | chr23 | 4431762 |
| chr18 | 3836919 | chr19 | 176442 | chr2 | 4476574 | chr20 | 10478809 | chr23 | 4450528 |
| chr18 | 4006499 | chr19 | 641527 | chr2 | 8721359 | chr20 | 10492440 | chr23 | 4459552 |
| chr18 | 4374709 | chr19 | 654262 | chr2 | 10113823 | chr20 | 10792667 | chr23 | 4968906 |
| chr18 | 4406681 | chr19 | 675336 | chr2 | 13471117 | chr20 | 10839762 | chr23 | 5160701 |
| chr18 | 4412830 | chr19 | 733523 | chr2 | 18074362 | chr20 | 12215709 | chr23 | 5361818 |
| chr23 | 5362705 | chr26 | 759385 | chr27 | 5188031 | chr3 | 4403395 | chr4 | 1793797 |
| chr23 | 5365790 | chr26 | 966956 | chr27 | 5193105 | chr3 | 7682833 | chr4 | 2073240 |
| chr23 | 5367234 | chr26 | 1123589 | chr27 | 5229133 | chr3 | 9367365 | chr4 | 2334190 |
| chr23 | 5378505 | chr26 | 1171393 | chr27 | 5273739 | chr3 | 9367622 | chr4 | 2334272 |
| chr23 | 5378526 | chr26 | 1191508 | chr27 | 5288145 | chr3 | 10390090 | chr4 | 2334294 |
| chr23 | 5475178 | chr26 | 1241159 | chr28 | 1059178 | chr3 | 10639985 | chr4 | 2334304 |
| chr24 | 215454 | chr26 | 1282165 | chr28 | 1178166 | chr3 | 12503190 | chr4 | 2334324 |
| chr24 | 335496 | chr26 | 1761825 | chr28 | 1237368 | chr3 | 16062066 | chr4 | 2412240 |
| chr24 | 349059 | chr26 | 1761830 | chr28 | 1326586 | chr3 | 16404163 | chr4 | 3686656 |
| chr24 | 368137 | chr26 | 1761866 | chr28 | 1326638 | chr3 | 16574228 | chr4 | 4396898 |
| chr24 | 433801 | chr26 | 2492665 | chr28 | 1434639 | chr3 | 18339074 | chr4 | 4726109 |
| chr24 | 946526 | chr26 | 2650784 | chr28 | 1569187 | chr3 | 27455275 | chr4 | 5298330 |
| chr24 | 1700627 | chr26 | 2674118 | chr28 | 1718605 | chr3 | 30324481 | chr4 | 5301249 |
| chr24 | 4314195 | chr26 | 3150150 | chr28 | 1798672 | chr3 | 34935448 | chr4 | 8271146 |
| chr24 | 4437133 | chr26 | 3153655 | chr28 | 2163542 | chr3 | 50570992 | chr4 | 10712067 |
| chr24 | 4836258 | chr26 | 3454288 | chr28 | 2220075 | chr3 | 55938039 | chr4 | 11263941 |
| chr24 | 4885733 | chr26 | 4370487 | chr28 | 2279488 | chr3 | 62923346 | chr4 | 12339481 |
| chr24 | 5075059 | chr26 | 4370521 | chr28 | 2384900 | chr3 | 62923348 | chr4 | 12499792 |
| chr24 | 5121984 | chr26 | 4449038 | chr28 | 2443299 | chr3 | 62923430 | chr4 | 14248495 |
| chr24 | 5200173 | chr26 | 4629268 | chr28 | 2578092 | chr3 | 75510563 | chr4 | 16513260 |
| chr24 | 5292824 | chr26 | 4824073 | chr28 | 2578135 | chr3 | 76465818 | chr4 | 29450515 |
| chr24 | 5315313 | chr26 | 4832264 | chr28 | 2714857 | chr3 | 82511066 | chr4 | 29670713 |
| chr24 | 5347096 | chr26 | 4860258 | chr28 | 2715081 | chr3 | 88643929 | chr4 | 33955263 |
| chr24 | 5363545 | chr26 | 4883633 | chr28 | 2793737 | chr3 | 95083391 | chr4 | 38140467 |
| chr24 | 5404237 | chr26 | 4907074 | chr28 | 2940168 | chr3 | 96864721 | chr4 | 39639936 |
| chr24 | 5620006 | chr26 | 4984911 | chr28 | 2959243 | chr3 | 104937361 | chr4 | 39795749 |
| chr25 | 269858 | chr27 | 1421190 | chr28 | 3066444 | chr3 | 104939759 | chr4 | 40106316 |
| chr25 | 1349368 | chr27 | 1544412 | chr28 | 3069648 | chr3 | 104992858 | chr4 | 46759694 |
| chr25 | 1385853 | chr27 | 1556830 | chr28 | 3133278 | chr3 | 105024153 | chr4 | 49891472 |
| chr25 | 1408131 | chr27 | 1659238 | chr28 | 3134236 | chr3 | 105026119 | chr4 | 50079787 |
| chr25 | 1417643 | chr27 | 1757837 | chr28 | 3143610 | chr3 | 105026234 | chr4 | 52986389 |
| chr25 | 1616881 | chr27 | 1777867 | chr28 | 3164537 | chr3 | 105391932 | chr4 | 57504076 |
| chr25 | 1682939 | chr27 | 1831436 | chr28 | 3266521 | chr3 | 105394067 | chr4 | 66101604 |
| chr25 | 1791259 | chr27 | 1831438 | chr28 | 3334886 | chr3 | 105642084 | chr4 | 66750658 |
| chr25 | 1870266 | chr27 | 2763081 | chr28 | 3344339 | chr3 | 107925261 | chr4 | 68821391 |
| chr25 | 1876695 | chr27 | 3158586 | chr28 | 3401492 | chr3 | 110294257 | chr4 | 79166320 |
| chr25 | 1886192 | chr27 | 3207099 | chr28 | 3560207 | chr3 | 110390054 | chr4 | 81350749 |
| chr25 | 2114798 | chr27 | 3336789 | chr28 | 3560209 | chr33 | 136125 | chr4 | 83042492 |
| chr25 | 2116204 | chr27 | 3406682 | chr28 | 3610524 | chr33 | 868744 | chr4 | 90670639 |
| chr25 | 2116212 | chr27 | 4027649 | chr28 | 3641199 | chr33 | 1120184 | chr4 | 91269069 |
| chr25 | 2350104 | chr27 | 4514498 | chr28 | 3668155 | chr33 | 1480259 | chr5 | 13625 |
| chr25 | 2490698 | chr27 | 4516803 | chr28 | 3886679 | chr33 | 1574811 | chr5 | 21090 |
| chr25 | 2671749 | chr27 | 4734803 | chr28 | 3901787 | chr33 | 1588456 | chr5 | 93130 |
| chr25 | 2697583 | chr27 | 4813757 | chr28 | 3902012 | chr33 | 1588458 | chr5 | 266411 |
| chr25 | 2734242 | chr27 | 4813794 | chr28 | 4352028 | chr4 | 535629 | chr5 | 14148661 |
| chr26 | 363161 | chr27 | 4878161 | chr3 | 3017205 | chr4 | 956663 | chr5 | 16013867 |
| chr26 | 391872 | chr27 | 4879792 | chr3 | 3227463 | chr4 | 1075519 | chr5 | 16908134 |
| chr26 | 566233 | chr27 | 4906960 | chr3 | 3376772 | chr4 | 1195945 | chr5 | 16920794 |
| chr26 | 616950 | chr27 | 5037719 | chr3 | 3675931 | chr4 | 1216927 | chr5 | 16982901 |
| chr26 | 646629 | chr27 | 5106792 | chr3 | 4398362 | chr4 | 1653010 | chr5 | 19078566 |
| chr5 | 21494150 | chr6 | 32311981 | chr8 | 15877559 | chr9 | 19755615 | | |
| chr5 | 21494155 | chr6 | 32353838 | chr8 | 20207487 | chr9 | 22098576 | | |
| chr5 | 21833084 | chr6 | 32373546 | chr8 | 20522394 | chr9 | 22139770 | | |
| chr5 | 23114829 | chr6 | 32376304 | chr8 | 20523865 | chr9 | 22539712 | | |
| chr5 | 24369896 | chr6 | 32383406 | chr8 | 20527810 | chr9 | 23425142 | | |
| chr5 | 25872954 | chr6 | 34155392 | chr8 | 20528359 | chr9 | 23463957 | | |
| chr5 | 27193394 | chr6 | 34896940 | chr8 | 20530302 | chr9 | 23463978 | | |
| chr5 | 27440098 | chr7 | 197081 | chr8 | 20533547 | chr9 | 23539310 | | |
| chr5 | 28371965 | chr7 | 3072964 | chr8 | 20627201 | chr9 | 23755071 | | |
| chr5 | 30029723 | chr7 | 3302420 | chr8 | 20639863 | chrLGE64 | 695854 | | |
| chr5 | 36278778 | chr7 | 10588608 | chr8 | 20733573 | chrUn_NT_464128v1 | 80351 | | |
| chr5 | 36428260 | chr7 | 10736707 | chr8 | 20766470 | chrUn_NT_464128v1 | 80888 | | |
| chr5 | 38156154 | chr7 | 11129143 | chr8 | 20863089 | chrUn_NT_464137v1 | 32520 | | |
| chr5 | 38331132 | chr7 | 12262422 | chr8 | 21099424 | chrUn_NT_464137v1 | 32526 | | |
| chr5 | 39293923 | chr7 | 12741388 | chr8 | 21107539 | chrUn_NT_464137v1 | 32532 | | |
| chr5 | 49728661 | chr7 | 16838143 | chr8 | 21208441 | chrUn_NT_464139v1 | 45486 | | |
| chr5 | 50233120 | chr7 | 22203954 | chr8 | 21597804 | chrUn_NT_464141v1 | 25111 | | |
| chr5 | 52119294 | chr7 | 22204056 | chr8 | 24561574 | chrUn_NT_464141v1 | 25117 | | |
| chr5 | 52997616 | chr7 | 22217126 | chr8 | 24878305 | chrUn_NT_464144v1 | 14257 | | |
| chr5 | 53937018 | chr7 | 22222428 | chr8 | 25249151 | chrUn_NT_464165v1 | 28684 | | |
| chr5 | 54221077 | chr7 | 22231914 | chr8 | 25255677 | chrUn_NT_464166v1 | 25170 | | |
| chr5 | 54482846 | chr7 | 22357340 | chr8 | 25324493 | chrUn_NT_464176v1 | 28927 | | |
| chr5 | 58235927 | chr7 | 22384841 | chr8 | 25324524 | chrUn_NT_464176v1 | 28936 | | |
| chr5 | 58240304 | chr7 | 22414534 | chr8 | 25347415 | chrUn_NT_464186v1 | 6536 | | |
| chr5 | 58704095 | chr7 | 22511684 | chr8 | 27552394 | chrUn_NT_464186v1 | 10237 | | |
| chr5 | 59140121 | chr7 | 22760940 | chr8 | 27552407 | chrUn_NT_464186v1 | 25090 | | |
| chr6 | 8372857 | chr7 | 22761366 | chr8 | 28095863 | chrUn_NT_464188v1 | 25831 | | |
| chr6 | 11461940 | chr7 | 22865043 | chr8 | 28653133 | chrUn_NT_464199v1 | 28290 | | |
| chr6 | 11507729 | chr7 | 25717871 | chr8 | 29363057 | chrUn_NT_464219v1 | 10827 | | |
| chr6 | 11909490 | chr7 | 25717875 | chr8 | 29384547 | chrUn_NT_464257v1 | 16583 | | |
| chr6 | 14574217 | chr7 | 26104560 | chr9 | 881362 | chrUn_NT_464263v1 | 22298 | | |
| chr6 | 17130648 | chr7 | 26104573 | chr9 | 914902 | chrUn_NT_464300v1 | 9335 | | |
| chr6 | 20107781 | chr7 | 27270579 | chr9 | 4339335 | chrUn_NT_464478v1 | 3238 | | |
| chr6 | 20558090 | chr7 | 27294957 | chr9 | 4448222 | chrUn_NT_464639v1 | 2596 | | |
| chr6 | 22136569 | chr7 | 34963494 | chr9 | 4868658 | chrUn_NT_464639v1 | 20173 | | |
| chr6 | 22315955 | chr7 | 36355191 | chr9 | 5117318 | chrUn_NT_464639v1 | 20197 | | |
| chr6 | 22395518 | chr8 | 2549725 | chr9 | 5126097 | chrUn_NT_464686v1 | 17798 | | |
| chr6 | 22402430 | chr8 | 2662453 | chr9 | 7925956 | chrUn_NT_464713v1 | 6601 | | |
| chr6 | 22492997 | chr8 | 3679867 | chr9 | 8283028 | chrUn_NT_464793v1 | 17113 | | |
| chr6 | 22650168 | chr8 | 3897179 | chr9 | 10685597 | chrUn_NT_465127v1 | 4108 | | |
| chr6 | 22817920 | chr8 | 3920338 | chr9 | 11926083 | chrUn_NT_465323v1 | 2602 | | |
| chr6 | 23063211 | chr8 | 4265400 | chr9 | 15011351 | chrUn_NT_465329v1 | 4891 | | |
| chr6 | 23101775 | chr8 | 4309191 | chr9 | 15015219 | chrUn_NT_466208v1 | 679 | | |
| chr6 | 23102058 | chr8 | 5176128 | chr9 | 15614928 | chrUn_NT_466381v1 | 4434 | | |
| chr6 | 23140346 | chr8 | 5320269 | chr9 | 15901828 | chrUn_NT_467238v1 | 5627 | | |
| chr6 | 23334727 | chr8 | 6007864 | chr9 | 15927987 | chrUn_NT_467270v1 | 2255 | | |
| chr6 | 26000212 | chr8 | 6121243 | chr9 | 15993232 | chrUn_NT_467639v1 | 7221 | | |
| chr6 | 31713799 | chr8 | 6642124 | chr9 | 16083698 | chrUn_NT_468148v1 | 2968 | | |
| chr6 | 32154878 | chr8 | 14714425 | chr9 | 16095646 | chrUn_NT_468606v1 | 5809 | | |
| chr6 | 32311804 | chr8 | 15118321 | chr9 | 17134844 | chrUn_NT_468859v1 | 8016 | | |
| chrUn_NT_463192v1 | 6463 | | | | | | | | |
| chrUn_NT_469922v1 | 7795 | | | | | | | | |
| chrUn_NT_470291v1 | 3988 | | | | | | | | |
| chrUn_NT_470291v1 | 3990 | | | | | | | | |
| chrUn_NT_471459v1 | 2820 | | | | | | | | |
| chrUn_NT_471567v1 | 2825 | | | | | | | | |
| chrUn_NT_471607v1 | 2107 | | | | | | | | |
| chrUn_NT_471607v1 | 2113 | | | | | | | | |
| chrUn_NT_471634v1 | 6625 | | | | | | | | |
| chrUn_NT_471760v1 | 2359 | | | | | | | | |
| chrUn_NT_471805v1 | 5471 | | | | | | | | |
| chrUn_NT_471827v1 | 319 | | | | | | | | |
| chrUn_NT_472117v1 | 1247 | | | | | | | | |
| chrUn_NT_472163v1 | 6453 | | | | | | | | |
| chrUn_NT_472519v1 | 766 | | | | | | | | |
| chrUn_NT_472519v1 | 899 | | | | | | | | |
| chrUn_NT_473083v1 | 4818 | | | | | | | | |
| chrUn_NT_474116v1 | 3220 | | | | | | | | |
| chrUn_NT_474208v1 | 4520 | | | | | | | | |
| chrUn_NT_474430v1 | 2433 | | | | | | | | |
| chrUn_NT_474430v1 | 4414 | | | | | | | | |
| chrUn_NT_474562v1 | 1864 | | | | | | | | |
| chrUn_NT_474562v1 | 2558 | | | | | | | | |
| chrUn_NT_474687v1 | 3076 | | | | | | | | |
| chrUn_NT_475266v1 | 1196 | | | | | | | | |
| chrUn_NT_475899v1 | 2898 | | | | | | | | |
| chrUn_NT_475899v1 | 2902 | | | | | | | | |
| chrUn_NT_479352v1 | 619 | | | | | | | | |
| chrUn_NT_479454v1 | 672 | | | | | | | | |
| chrUn_NT_479497v1 | 470 | | | | | | | | |
| chr16_NT_462548v1_random | 3467 | | | | | | | | |
| chr1_NT_456103v1_random | 3063 | | | | | | | | |
| chr1_NT_457825v1_random | 369 | | | | | | | | |
| chr22_NT_463008v1_random | 6888 | | | | | | | | |
| chr25_NT_463213v1_random | 6611 | | | | | | | | |
| chr2_NT_457893v1_random | 15417 | | | | | | | | |
| chr30_NT_463422v1_random | 41922 | | | | | | | | |
| chr32_NT_463446v1_random | 1750 | | | | | | | | |
| chr32_NT_463446v1_random | 5275 | | | | | | | | |
| chr33_NT_463454v1_random | 73366 | | | | | | | | |
| chr4_NT_459800v1_random | 331914 | | | | | | | | |
| chr4_NT_450801v1_random | 13745 | | | | | | | | |
| chr4_NT_459801v1_random | 24225 | | | | | | | | |
| chr4_NT_459802v1_random | 45509 | | | | | | | | |
| chr4_NT_459802v1_random | 51182 | | | | | | | | |
| chr4_NT_459602v1_random | 60221 | | | | | | | | |
| chr4_NT_459802v1_random | 60517 | | | | | | | | |
| chr4_NT_459802v1_random | 104628 | | | | | | | | |
| chr4_NT_459802v1_random | 116555 | | | | | | | | |
| chr4_NT_459802v1_random | 120297 | | | | | | | | |

### Results

Plotting of the first two principal components of the CpGs before and after differential methylation analysis reveal meaningful clustering of the samples. Differentially methylated Positions (DMPs) were able to effectively cluster of the different locations in a meaningful way.

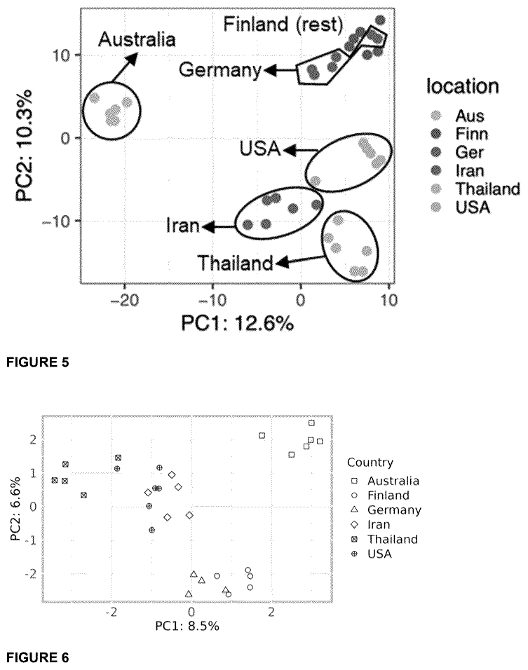

Plotting of the first two principal components of the differential methylation analysis CpGs reveal meaningful clustering of the samples (FIG. 5).

PCA plots was run to differentiate between the different locations using the Beadchip data (FIG. 6).

In summary, plotting of the first two principal components of the CpGs reveal meaningful clustering of the samples processed by sequencing and beadchip data. CpG sites were able to effectively cluster of the different locations in a meaningful way. The results of the origin traceability application with the beadchip were shown to be replicable.

1. A method of detecting DNA methylation and/or determining a test methylation profile from genomic material contained in a biological sample obtained from a test animal-derived product, the method comprises the step of:
contacting a genomic material sample from the test animal-derived product with a DNA methylation array specific for species of the test animal,
wherein the test animal is a monogastric livestock.

2. A method for the identification of the geographic origin of a test animal-derived product, the method comprising the steps of:
(a) carrying out the method of claim 1 to determine a test methylation profile of the test animal-derived product; and
(b) comparing the test methylation profile determined in (a) with one or more predetermined reference methylation profiles, wherein each of the one or more predetermined reference methylation profiles is specific for a distinct geographic origin of subjects which are of the same biological taxon as the test animal;
wherein if the test methylation profile is significantly similar to one of the predetermined reference methylation profiles, the test animal-derived product has a geographical origin similar to the subjects of the predetermined reference methylation profile; and/or
wherein if the test methylation profile is different to one of the predetermined reference methylation profiles, the test animal-derived product has a geographical origin different to the subjects of the predetermined reference methylation profile.
wherein the test animal is a monogastric livestock.

3. The method according to claim 2, wherein the distinct geographic origin is a geographic location that is considered to be the habitat, where the test animal, was birthed, hatched and/or reared, or at least reared for a significant time during their lifetime.

4. An in vitro method for predicting the biological age of a test animal from which a product is derived, the method comprising the step of:
(a) carrying out the method of claim 1 to determine a test methylation profile of the test animal; and
(b) comparing the test methylation profile from (a) with the methylation profile from an age-correlated reference sample, thereby establishing the epigenetic age and predicting the biological age of the test animal from which the product is derived; and
wherein the test animal is a monogastric livestock.

5. A method of determining if a test animal from which a product is derived has been treated and/or is currently undergoing treatment with at least one antibiotic and/or veterinary chemical, the method comprising:
(a) carrying out the method of claim 1 to determine a test methylation profile of the test animal; and
(b) comparing the test methylation profile obtained from (a) with a reference methylation profile obtained from a control animal or a control animal-derived product, where the control animal was not treated and/or is not currently undergoing treatment with at least one antibiotic and/or veterinary chemical,
wherein a difference in the test methylation profile of (a) compared to the reference methylation profile from the control animal, is indicative of the test animal having been treated and/or is currently undergoing treatment with at least one antibiotic and/or veterinary chemical; and/or
wherein a significant similarity in the test methylation profile of (a) compared to the reference methylation profile, is indicative of the test animal having not been treated and/or is currently not undergoing treatment with at least one antibiotic and/or veterinary chemical; and
wherein the test animal is a monogastric livestock.

6. A method of determining if a test animal from which a product is derived has been treated and/or is currently undergoing treatment with at least one antibiotic, and if so, determining the distinct class of antibiotics with which the test animal is being treated and/or is currently undergoing treatment, the method comprising:
(a) carrying out the method of claim 1 to determine a test methylation profile of the test animal; and
(b) comparing the test methylation profile obtained from (a) with one or more predetermined reference methylation profiles, wherein each of the predetermined reference methylation profiles is from a different animal of the same biological taxon as the test animal, and each of the different animals was treated with a different class of antibiotics,
wherein if the test methylation profile of (a) is significantly similar to one of the predetermined reference methylation profiles, the test animal from which the product is derived is confirmed for being treated with the same class of antibiotics with which the animal with the predetermined reference profile is treated with and the test animal has been confirmed for having been treated and/or is currently undergoing treatment with the distinct class of antibiotics; and/or
wherein if the test methylation profile of (a) is significantly different to one of the predetermined reference methylation profiles, the test animal from which the product is derived is confirmed for not being treated with the same class of antibiotics with which the animal with the predetermined reference profile is treated with and the test animal has not been confirmed for having been treated and/or is currently undergoing treatment with the distinct class of antibiotics; and
wherein the test animal is a monogastric livestock.

7. The method according to claim 5, wherein
the distinct classes of antibiotics are amphenicols, aminocyclitols, aminoglycosides, ansamycins, beta-lactams, carbaephem, carbapenems, cephalosporins, chloramphenicol, fluoroquinolones, glycopeptides, glycylcyclines, ketolides, lincosamides, lipopeptides, macrolides, monobactams, nitrofurans, nitroimidazoles, oxazolidinones, penicillins, phosphorus containing antibiotics; and/or
the veterinary chemical is an anti-parasitic, an anti-viral, a feed additive, a disinfectant, glutaraldehyde, and/or formalin.

8. A method of certifying a test animal-derived product sample, the method comprising the steps of:
(a) contacting genomic material contained in a biological sample from the product derived from the test animal with a DNA methylation-based array to determine a test methylation profile of the test animal; and
(b) comparing the test methylation profile obtained from (a) with a reference methylation profile obtained from a control animal of the same biological taxon of the test animal from which the product sample is derived from, where the control animal is not slaughtered by a single cut across the throat severing both carotid arteries, both jugular veins, both vagus nerves, trachea and/or esophagus and/or the control animal is not bled to death
wherein a difference in the test methylation profile of (a) compared to the reference methylation profile from the control animal, is indicative of the test animal having been slaughtered by a single cut across the throat severing both carotid arteries, both jugular veins, both vagus nerves, trachea and/or esophagus and/or of the test animal having been bled to death and the test animal-derived product is certified so; and/or
wherein a significant similarity in the test methylation profile of (a) compared to the reference methylation profile from the control animal, is indicative of the test animal not having been slaughtered by a single cut across the throat severing both carotid arteries, both jugular veins, both vagus nerves, trachea and/or esophagus and/or of the test animal having been bled to death and the test animal-derived product is certified so; and
wherein the test animal is a monogastric livestock.

9. A method of determining the supplier from which a test animal-derived product sample originates, the method comprising the steps of:
(a) carrying out the method of claim 1, to determine a test methylation profile of the test animal-derived product; and
(b) comparing the test methylation profile determined in (a) with a panel of predetermined reference methylation profiles of the same biological taxon of the test animal from which the product sample derives, wherein each of the predetermined reference methylation profiles is from a different supplier,
wherein if the test methylation profile of (a) is significantly similar to one of the predetermined reference methylation profiles, the test animal-derived product sample is confirmed of originating from a first supplier from which a first reference animal with the predetermined reference profile is obtained; and/or
wherein if the test methylation profile of (a) is different to one of the predetermined reference methylation profiles, the test animal-derived product sample is confirmed of not originating from a first supplier from which a first reference animal with the predetermined reference profile is obtained; and
wherein the test animal is a monogastric livestock.

10. A method of certifying a test animal-derived product sample, the method comprising the steps of:
(a) carrying out the method of claim 1 to determine a test methylation profile of the test animal; and
(b) comparing the test methylation profile obtained from (a) with a reference methylation profile obtained from a control animal of the same biological taxon of the test animal from which the product sample is derived from, where the control animal is bred under a known distinct type of animal husbandry,
wherein a significant similarity in the test methylation profile of (a) compared to the reference methylation profile from the control animal, is indicative of the test animal having been bred under the same distinct type of animal husbandry as the control animal and the test animal-derived product is certified so; and/or
wherein a difference in the test methylation profile of (a) compared to the reference methylation profile of the control animal, is indicative of the test animal having been bred under another distinct type of animal husbandry as the control animal; and
wherein the test animal is a monogastric livestock.

11. A method of determining if a test animal derived product sample is derived from an animal that is suitable for selective breeding, the method comprising the steps of:
(a) carrying out the method of claim 1 to determine a test methylation profile of the test animal; and
(b) comparing the test methylation profile obtained from (a) with a reference methylation profile obtained from a control animal of the same biological taxon of the test animal from which the product sample is derived from, where the control animal comprises at least one phenotype of interest for selective breeding,
wherein a significant similarity in the test methylation profile of (a) compared to the reference methylation profile from the control animal, is indicative of the test animal being suitable for selective breeding; and/or
wherein a difference in the test methylation profile of (a) compared to the reference methylation profile of the control animal, is indicative of the test animal not being suitable for selective breeding; and
wherein the test animal is a monogastric livestock.

12. The method according to claim 1, wherein the monogastric livestock includes terrestrial and aquatic livestock.

13. The method according to claim 1, wherein the monogastric livestock is selected from the group consisting of pig, horse, donkey, rabbit and mule and/or poultry and vertebrate fish which is selected from the group consisting of chicken, turkey, duck, goose, and quail.

14. The method according to claim 1, wherein the animal-derived product is meat, muscle, at least one organ, milk, collagen, feather, blood and/or bone.

15. A DNA methylation-based array for carrying out the method according to claim 1.
